(19) United States
(12) Patent Application Publication
(10) Pub. No.: US 2004/0152072 A1
(43) Pub. Date: Aug. 5, 2004

(54) Reverse transcription

(75) Inventor: Gary F. Gerard, Frederick, MD (US)

Correspondence Address: STERNE, KESSLER, GOLDSTEIN & FOX PLLC, 1100 NEW YORK AVENUE, N.W., WASHINGTON, DC, 20005, US

(73) Assignee: Invitrogen Corporation

(21) Appl. No.: 10/629,859

(22) Filed: Jul. 30, 2003

Related U.S. Application Data
(60) Provisional application No. 60/399,152, filed on Jul. 30, 2002.
(60) Provisional application No. 60/463,037, filed on Apr. 16, 2003.

(51) Int. Cl. C12Q 1/70; C12Q 1/68; C12N 9/99

(52) U.S. Cl. 435/5; 435/6; 435/184

(57) ABSTRACT

The present invention relates generally to inhibitors useful to prevent, reduce, substantially reduce or eliminate degradation of nucleic acid templates during nucleic acid synthesis. Such inhibitors of the invention preferably prevent adverse chemical reactions during reverse transcription, which result in degradation of RNA molecules, especially messenger RNA (MRNA) molecules. The invention also relates to compositions comprising inhibitors of the invention, and to methods of producing, amplifying, or sequencing nucleic acid molecules (particularly cDNA molecules) using these compositions, particularly at elevated temperatures. The invention also relates to nucleic acid molecules produced by these methods, to vectors and host cells comprising these cDNA molecules and to the use of such nucleic acid molecules to produce desired polypeptides. The invention further relates to kits comprising such inhibitors or compositions.

### CROSS-REFERENCE TO RELATED APPLICATIONS

The present application claims priority to U.S. Provisional Application No. 60/399,152, filed Jul. 30, 2002, the disclosure of which is entirely incorporated herein by reference.

### BACKGROUND OF THE INVENTION

1. Field of the Invention

The present invention is in the fields of molecular and cellular biology. The invention is generally related to one or more inhibitors of nucleic acid (e.g.RNA) degradation and their use in the reverse transcription of nucleic acid molecules, especially messenger RNA molecules. Specifically, the invention relates to compounds or molecules or compositions having the capability of preventing, reducing, substantially reducing, or eliminating RNA degradation during nucleic acid synthesis and methods of producing, amplifying or sequencing nucleic acid molecules (particularly cDNA molecules) using these inhibitors. The invention also relates to nucleic acid molecules produced by these methods and to the use of such nucleic acid molecules to produce desired polypeptides. The invention also concerns kits or compositions comprising one or more or a combination of such inhibitors. The invention also relates to reaction mixtures comprising one or more or a combination of these inhibitors.

2. Related Art

### cDNA and cDNA Libraries

In examining the structure and physiology of an organism, tissue or cell, it is often desirable to determine its genetic content. The genetic framework of an organism is encoded in the double-stranded sequence of nucleotide bases in the deoxyribonucleic acid (DNA) which is contained in the somatic and germ cells of the organism. The genetic content of a particular segment of DNA, or gene, is typically manifested upon production of the protein which the gene encodes. In order to produce a protein, a complementary copy of one strand of the DNA double helix is produced by RNA polymerase enzymes, resulting in a specific sequence of ribonucleic acid (RNA). This particular type of RNA, since it contains the genetic message from the DNA for production of a protein, is called messenger RNA (mRNA).

Within a given cell, tissue or organism, there exist myriad mRNA species, each encoding a separate and specific protein. This fact provides a powerful tool to investigators interested in studying genetic expression in a tissue or cell. mRNA molecules may be isolated and further manipulated by various molecular biological techniques, thereby allowing the elucidation of the full functional genetic content of a cell, tissue or organism.

One common approach to the study of gene expression is the production of complementary DNA (cDNA) clones. In this technique, the mRNA molecules from an organism are isolated from an extract of the cells or tissues of the organism. This isolation often employs solid chromatography matrices, such as cellulose or agarose, to which oligomers of thymidine (T) have been complexed. Since the 3′ termini on most eukaryotic mRNA molecules contain a string of adenosine (A) bases, and since A base pairs with T, the mRNA molecules can be rapidly purified from other molecules and substances in the tissue or cell extract. From these purified mRNA molecules, cDNA copies may be made using the enzyme reverse transcriptase (RT), which results in the production of single-stranded complementary DNA or cDNA molecules. This reaction is typically referred to as the first strand reaction. The single-stranded cDNAs may then be converted into a complete double-stranded DNA copy (i.e., a double-stranded cDNA) of the original mRNA (and thus of the original double-stranded DNA sequence, encoding this mRNA, contained in the genome of the organism) by the action of a DNA polymerase. The protein-specific double-stranded cDNAs can then be inserted into a plasmid or viral vector, which is then introduced into a host bacterial, yeast, animal or plant cell. The host cells are then grown in culture media, resulting in a population of host cells containing (or in many cases, expressing) the gene of interest.

This entire process, from isolation of mRNA from a source organism or tissue to insertion of the cDNA into a plasmid or vector to growth of host cell populations containing the isolated gene, is termed “cDNA cloning.” The set or population of cDNAs prepared from a given source of mRNAs or population of mRNA molecules is called a “cDNA library.” The cDNA clones in a cDNA library correspond to the genes transcribed in the source tissue. Analysis of a cDNA library can yield much information on the pattern of gene expression in the organism or tissue from which it was derived.

### Retroviral Reverse Transcriptase Enzymes

Three prototypical forms of retroviral reverse transcriptase have been studied thoroughly. Moloney Murine Leukemia Virus (M-MLV) reverse transcriptase contains a single subunit of 78 kDa with RNA-dependent DNA polymerase and RNase H activity. This enzyme has been cloned and expressed in a fully active form in E. coli (reviewed in Prasad, V. R., Reverse Transcriptase, Cold Spring Harbor, N.Y.: Cold Spring Harbor Laboratory Press, p.135 (1993)). Human Immunodeficiency Virus (HIV) reverse transcriptase is a heterodimer of p66 and p51 subunits in which the smaller subunit is derived from the larger by proteolytic cleavage. The p66 subunit has both a RNA-dependent DNA polymerase and an RNase H domain, while the p51 subunit has only a DNA polymerase domain. Active HIV p66/p51 reverse transcriptase has been cloned and expressed successfully in a number of expression hosts, including E. coli (reviewed in Le Grice, S. F. J., Reverse Transcriptase, Cold Spring Harbor, N.Y.: Cold Spring Harbor Laboratory press, p. 163 (1993)). Within the HIV p66/p51 heterodimer, the 51-kD subunit is catalytically inactive, and the 66-kD subunit has both DNA polymerase and RNase H activity (Le Grice, S. F. J., et al., EMBO Journal 10:3905 (1991); Hostomsky, Z., et al., J. Virol. 66:3179 (1992)). Avian Sarcoma-Leukosis Virus (ASLV) reverse transcriptase, which includes but is not limited to Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase, Avian Sarcoma Virus Y73 Helper Virus YAV reverse transcriptase, Rous Associated Virus (RAV) reverse transcriptase, and Myeloblastosis Associated Virus (MAV) reverse transcriptase, is also a heterodimer of two subunits, α (approximately 62 kDa) and β (approximately 94 kDa), in which α is derived from β by proteolytic cleavage (reviewed in Prasad, V. R., Reverse Transcriptase, Cold Spring Harbor, N.Y.: Cold Spring Harbor Laboratory Press (1993), p. 135). ASLV reverse transcriptase can exist in two additional catalytically active structural forms, ββ and α (Hizi, A. and Joklik, W. K., J. Biol. Chem. 252: 2281 (1977)). Sedimentation analysis suggests αβ and ββ are dimers and that the a form exists in an equilibrium between monomeric and dimeric forms (Grandgenett, D. P., et al., Proc. Nat. Acad. Sci. USA 70:230 (1973); Hizi, A. and Joklik, W. K., J. Biol. Chem. 252:2281 (1977); and Soltis, D. A. and Skalka, A. M., Proc. Nat. Acad. Sci. USA 85:3372 (1988)). The ASLV αβ and ββ reverse transcriptases are the only known examples of retroviral reverse transcriptase that include three different activities in the same protein complex: DNA polymerase, RNase H, and DNA endonuclease (integrase) activities (reviewed in Skalka, A. M., Reverse Transcriptase, Cold Spring Harbor, N.Y.: Cold Spring Harbor Laboratory Press (1993), p. 193). The a form lacks the integrase domain and activity.

Various forms of the individual subunits of ASLV reverse transcriptase have been cloned and expressed. These include a 98-kDa precursor polypeptide that is normally processed proteolytically to β and a 4 kDa polypeptide removed from the β carboxy end (Alexander, F., et al., J. Virol. 61:534 (1987) and Anderson, D. et al., Focus 17:53 (1995)), and the mature β subunit (Weis, J. H. and Salstrom, J. S., U.S. Pat. No. 4,663,290 (1987); and Soltis, D. A. and Skalka, A. M., Proc. Nat. Acad. Sci. USA 85:3372 (1988)). (See also Werner S. and Wohrl B. M., Eur. J. Biochem. 267:4740-4744 (2000); Werner S. and Wohrl B. M., J. Virol. 74:3245-3252 (2000); Werner S. and Wohrl B. M., J. Biol. Chem. 274:26329-26336 (1999).) Heterodimeric RSV αβ reverse transcriptase has also been purified from E. coli cells expressing a cloned RSV β gene (Chernov, A. P., et al., Biomed. Sci. 2:49 (1991)).

### Reverse Transcription Efficiency

As noted above, the conversion of MRNA into cDNA by reverse transcriptase-mediated reverse transcription is an essential step in the study of proteins expressed from cloned genes. However, the use of unmodified reverse transcriptase to catalyze reverse transcription is inefficient for a number of reasons. First, reverse transcriptase sometimes degrades an RNA template before the first strand reaction is initiated or completed, primarily due to the intrinsic RNase H activity present in reverse transcriptase. In addition, while secondary structure of the MRNA molecule itself may make some mRNAs refractory to first strand synthesis.

Removal of the RNase H activity of reverse transcriptase can eliminate the first problem and improve the efficiency of reverse transcription (Gerard, G. F., et al., FOCUS 11(4):60 (1989); Gerard, G. F., et al., FOCUS 14(3):91 (1992)) and U.S. Pat. Nos. 5,244,797, 5,404,776, 5,668,005, 6,063,608. However reverse transcriptases which lack RNase H activity (“RNase H minus” forms) do not address the additional problems of mRNA secondary structure.

Secondary structure of RNA molecules can adversely affect the efficiency of reverse transcription by acting as a structural barrier to prevent the reverse transcriptase from copying the entire RNA templates during cDNA synthesis. Such secondary structures can form, for example, when regions of RNA molecules have sufficient complementarity to hybridize and form double stranded RNA. Generally, the formation of RNA secondary structures can be reduced by raising the temperature of solutions which contain the RNA molecules. Thus, in many instances, it is desirable to reverse transcribe RNA at temperatures above 37° C. because the efficiency and specificity of cDNA synthesis from MRNA catalyzed are improved.

However, higher reaction temperatures may also accelerate the rate of degradation of the very templates being copied during the reverse transcriptase reaction. For example, metal ion-catalyzed hydrolysis of RNA can take place during reverse transcription. At temperatures above 37° C. (AbouHaidar et al., Z. Naturforsch 54, 542-548, 1999) and at pH 7.5 and above in Tris-HCl buffers (Barshevskaya et al., Molekulyarnaya Biologiya 21, 1235-1241, 1987), the rate of RNA breakdown is directly proportional to Mg²⁺ concentration between 1 and 10 mM. Free Mg²⁺ (1 to 3.5 mM) and alkaline pH (8.4) are present during cDNA synthesis with murine and avian retroviral RT's creating conditions under which Mg²⁺-catalyzed RNA breakdown occurs. (Gerard et al., Molecular Biotechnology 8, 61-77, 1997; Krug et al., Methods Enzymol. 152, 316-325, 1987; Schwabe et al., FOCUS 20, 30-33, 1999)

### SUMMARY OF THE INVENTION

The present invention satisfies the need discussed above. The present invention provides a method for inhibiting, preventing, reducing, substantially reducing or eliminating nucleic acid degradation. In a preferred aspect, the invention provides inhibitors, compositions, and reaction mixtures comprising such inhibitors, and methods useful in overcoming limitations of reverse transcription of nucleic acid molecules (particularly cDNA molecules). More specifically, the invention relates to preventing, reducing, substantially reducing, or eliminating degradation of RNA (especially mRNA) during reverse transcription via use of one or a combination of these inhibitory molecules.

In general, the invention provides compositions or reaction mixtures for use in reverse transcription of a nucleic acid molecule comprising one or more (e.g., one, two, three, four, five, ten, fifteen, etc.) inhibitors of nucleic acid degradation. Such compositions or reaction mixtures may further comprise one or more components selected from the group consisting of one or more one or more polypeptides having reverse transcriptase activity (including reverse transcriptases and DNA polymerases), one or more nucleic acid templates (e.g. one or more or a population of RNA, mRNA, or poly A RNA molecules), one or more nucleotides, one or more suitable buffers or buffering salts, and one or more DNA polymerases. The compositions of the invention may also comprise one or more oligonucleotide primers, preferably oligo dT primers.

The inhibitors of the invention are capable of inhibiting degradation of RNA during reverse transcription of one or more nucleic acid molecules. In an aspect of the: invention, the inhibitor is not a ribonuclease, inhibitor such as RNaseOUT™ (Invitrogen Corporation), ANTI-RNase (Ambion), SUPERase.In™. (Ambion), or RNase Inhibitor (Roche). In another aspect of the invention, the inhibitors of the invention preferably interacts or binds covalently or non-covalently with one or more components that degrade the RNA molecules. Such interaction or binding may be based on interaction of the negatively charged inhibitor and positively charged degradation component and this interaction may be reversible or irreversible. In a preferred aspect of the invention, the inhibitor has a net negative charge and is selected from the group consisting of nucleotides, anions, basic salts, chelators and amino acids and the degradation component has a net positive charge and is selected from the group consisting of metal ions, cations, cationic salts, and amino acids.

In another aspect of the invention, the inhibitor is at a sufficient concentration or amount in a nucleic acid synthesis reaction to inhibit, prevent, reduce, substantially reduce or eliminate nucleic acid (e.g. RNA) degradation by a degradation component. Preferably, the concentration or amount of the inhibitor does not adversely affect or does not substantially adversely affect nucleic acid synthesis. In one embodiment, the degradation component is a component useful or required for nucleic acid synthesis but also degrades RNA in a nucleic acid synthesis reaction. For example, Mg⁺⁺ is a desired component of reverse transcriptase enzymes during nucleic acid synthesis. In such cases the inhibitor of the invention may be added in an amount or at a concentration sufficient to inhibit, prevent, reduce, substantially reduce, or eliminate nucleic acid degradation but not adversely affect or not substantially adversely affect nucleic acid synthesis. In one preferred aspect, the inhibitor reversibly interacts or binds the degradation component and thus the degradation component may be made available for nucleic acid synthesis but the availability of degradation component to degrade nucleic acid molecules is minimized. In one such embodiment, as the degradation component becomes available (for example, the inhibitor may dissociate from or release the degradation component) the degradation component can be utilized by the polymerase enzyme (eg. reverse transcriptase) to synthesize nucleic acid molecules from one or more templates. Thus, the degradation component becomes unavailable to degrade the nucleic acid template. Thus, the invention avoids the presence of excess degradation component in a nucleic acid synthesis reaction, which may result in degradation of the templates, but still allows the degradation component to be utilized as a desired component in the synthesis reaction. In another aspect, the inhibitor is a useful or desired component of nucleic acid synthesis and may include any one or a number of nucleotides.

The reverse transcriptases used in the invention may be any polypeptide or protein having reverse transcriptase activity. Examples include reverse transcriptase (such as retroviral RTs) and DNA polymerases having reverse transcriptase activity (such as pol I type DNA polymerases). Such reverse transcriptase enzymes are preferably thermostable DNA polymerases or reverse transcriptases. Such reverse transcriptases may be modified or mutated such that the thermostability of the enzyme is increased or enhanced. The reverse transcriptases may be single chained (single subunit) or multi-chained (multi-subunit) and may be reduced or substantially reduced or lacking in RNase H activity. Preferred reverse transcriptases for use in the invention may be selected from the group consisting of Moloney Murine Leukemia Virus (M-MLV) RNase H−reverse transcriptase, Rous Sarcoma Virus (RSV) reverse transcriptase, Avian Myeloblastosis Virus (AMV) reverse transcriptase, Rous Associated Virus (RAV) reverse transcriptase, Myeloblastosis Associated Virus (MAV) reverse transcriptase or other ASLV reverse transcriptases and Human Immunodeficiency Virus (HIV) reverse transcriptase and mutants thereof. Also preferred for use in the invention are thermostable DNA polymerases which exhibit reverse transcriptase activity. Examples include Tth DNA polymerases, Tne DNA polymerase, Tma DNA polymerase, Taq DNA polymerase and the like.

As noted above, enzymes used in the compositions of the invention include reverse transcriptases which exhibit reverse transcriptase activity either upon the formation of multimers (e.g., dimers) or as individual protein molecules (i.e., in monomeric form). Examples of reverse transcriptases which exhibit reverse transcriptase activity upon the formation of multimers include AMV, RSV and HIV reverse transcriptases. One example of a reverse transcriptase which exhibits reverse transcriptase activity as separate, individual proteins (i.e., in monomeric form) is M-MLV reverse transcriptase.

The invention is also directed to methods for making one or more nucleic acid molecules or a population of nucleic acid molecules, comprising mixing one or more nucleic acid templates (preferably one or more RNA templates and most preferably one or more messenger RNA templates or a population of mRNA templates) with one or more inhibitors (or inhibitor compositions) of the invention, and one or more reverse transcriptases (or polypeptides having reverse transcriptase activity) and incubating the mixture under conditions sufficient to-make a first nucleic acid molecule or molecules complementary to all or a portion of the one or more nucleic acid templates. Such conditions may include incubation with one or more primers (such as oligo dT primers), one or more buffers or buffering salts, and/or one or more nucleotides. Preferably, the nucleic acid synthesis method of the invention is conducted under condition sufficient to inhibit, prevent, reduce, substantially reduce or eliminate degradation of the nucleic acid template or templates prior to and/or during nucleic acid synthesis. In some embodiments, the mixture is incubated at an elevated temperature, i.e., greater than 37° C. In a preferred aspect, the incubation temperature is determined by the temperature optimum of the enzyme(s) used in nucleic acid synthesis and such incubation temperature is preferably at the optimum or within 5° C. above or below the optimum. In specific embodiments, the elevated temperature may be from about 40° C. or greater, from about 45° C. or greater, from about 50° C. or greater, from about 51° C. or greater, from about 52° C. or greater, from about 53° C. or greater, from about 54° C. or greater, from about 55° C. or greater, from about 56° C. or greater, from about 57° C. or greater, from about 58° C. or greater, from about 59° C. or greater, from about 60° C. or greater, from about 61° C. or greater, from about 62° C. or greater, from about 63° C. or greater, from about 64° C. or greater, from about 65° C. or greater, from about 66° C. or greater, from about 67° C. or greater, from about 68° C. or greater, from about 69° C. or greater, from about 70° C. or greater, from about 71° C. or greater, from about 72° C. or greater, from about 73° C. or greater, from about 74° C. or greater, from about 75° C. or greater, from about 76° C. or greater, from about 77° C. or greater, or from about 78° C. or greater, or from about 80° C. or greater. An elevated temperature may be within a temperature range of from about 40° C. to about 45° C., from about 40° C. to about 48° C., from about 40° C. to about 50° C., from about 40° C. to about 52° C., from about 40° C. to about 55° C., from about 40° C. to about 58° C., from about 40° C. to about 60° C., from about 40° C. to about 65° C., from about 42° C. to about 45° C., from about 42° C. to about 48° C., from about 42° C. to about 50° C., from about 42° C. to about 52° C., from about 42° C. to about 55° C., from about 42° C. to about 58° C., from about 42° C. to about 60° C., from about 42° C. to about 65° C., from about 45° C. to about 48° C., from about 45° C. to about 50° C., from about 45° C. to about 52° C., from about 45° C. to about 55° C., from about 45° C. to about 58° C., from about 45° C. to about 60° C., from about 45° C. to about 65° C., from about 48° C. to about 50° C., from about48° C. to about 52° C., from about 48° C. to about 55° C., from about 48° C. to about 58° C., from about 48° C. to about 60° C., from about 48° C. to about 65° C., from about 50° C. to about 52° C., from about 50° C. to about 55° C., from about 50° C. to about 58° C., from about 50° C. to about 60° C., from about 50° C. to about 65° C., from about 52° C. to about 55° C., from about 52° C. to about 58° C., from about 52° C. to about 60° C., from about 52° C. to about 65° C., from about 55° C. to about 58° C., from about 55° C. to about 60° C., from about 55° C. to about 65° C., from about 55° C. to about 70° C., from about 58° C. to about 60° C., from about 58° C. to about 65° C., from about 58° C. to about 70° C. An elevated temperature may be within a temperature range from about 37° C. to about 75° C., from about 40° C. to about 75° C., from about 45° C. to about 75° C., from about 50° C. to about 75° C., from about 51° C. to about 75° C., from about 52° C. to about 75° C., from about 53° C. to about 75° C., from about 54° C. to about 75° C., from about 55° C. to about 75° C. In other embodiments, the elevated temperature may be within the range of about 50° C. to about 70° C., from about 51° C. to about 70° C., from about 52° C. to about 70° C., from about 53° C. to about 70° C., from about 54° C. to about 70° C., from about 55° C. to about 70° C., from about 56° C. to about 65° C., from about 56° C. to about 64° C. or about 56° C. to about 62° C. In other embodiments, the elevated temperature may be within the range of about 46° C. to about 60° C., from about 47° C. to about 60° C., from about 49° C. to about 60° C., from about 51° C. to about 60° C., from about 53° C. to about 60° C., from about 54° C. to about 60° C., or about 60° C. to about 85° C. In additional specific embodiments, the first nucleic acid molecule is a single-stranded cDNA or population of single-stranded cDNA molecules.

Nucleic acid templates suitable for reverse transcription according to this aspect of the invention include any nucleic acid molecule or population of nucleic acid molecules (preferably RNA and most preferably mRNA), particularly those derived from a cell or tissue. In a specific aspect, a population of mRNA molecules (a number of different mRNA molecules, typically obtained from a particular cell or tissue type) is used to make a cDNA library, in accordance with the invention. Examples of cellular sources of nucleic acid templates include bacterial cells, fungal cells, plant cells and animal cells.

The invention also concerns methods for making one or more double-stranded nucleic acid molecules. Such methods comprise (a) mixing one or more nucleic acid templates (preferably RNA or mRNA, and more preferably a population of mRNA templates) with one or more inhibitors of the invention and one or more reverse transcriptases or polypeptides having reverse transcriptase activity; (b) incubating the mixture under conditions sufficient to make a first nucleic acid molecule or molecules complementary to all or a portion of the one or more templates; and (c) incubating the first nucleic acid molecule or molecules under conditions sufficient to make a second nucleic acid molecule or molecules complementary to all or a portion of the first nucleic acid molecule or molecules, thereby forming one or more double-stranded nucleic acid molecules comprising the first and second nucleic acid molecules. Such conditions may include incubation with one or more primers (such as oligo dT primers), one ore more DNA polymerases, one or more buffers or buffering salts, and/or one or more nucleotides. Preferably, the nucleic acid synthesis method of the invention is conducted under condition sufficient to inhibit, prevent, reduce, substantially reduce or eliminate degradation of the nucleic acid template or templates prior to and/or during nucleic acid synthesis. In some embodiments, the incubation of step (b) is performed at an elevated temperature. In a preferred aspect, the incubation temperature is determined by the temperature optimum of the enzyme(s) used in nucleic acid synthesis and such incubation temperature is preferably at the optimum or within 5° C. above or below the optimum. In specific embodiments, the elevated temperature may be from about 40° C. or greater, from about 45° C. or greater, from about 50° C. or greater, from about 51° C. or greater, from about 52° C. or greater, from about 53° C. or greater, from about 54° C. or greater, from about 55° C. or greater, from about 56° C. or greater, from about 57° C. or greater, from about 58° C. or greater, from about 59° C. or greater, from about 60° C. or greater, from about 61° C. or greater, from about 62° C. or greater, from about 63° C. or greater, from about 64° C. or greater, from about 65° C. or greater, from about 66° C. or greater, from about 67° C. or greater, from about 68° C. or greater, from about 69° C. or greater, from about 70° C. or greater, from about 71° C. or greater, from about 72° C. or greater, from about 73° C. or greater, from about 74° C. or greater, from about 75° C. or greater, from about 76° C. or greater, from about 77° C. or greater, from about 78° C. or greater, or from about 80° C. or greater. An elevated temperature may be within a temperature range of from about 40° C. to about 45° C., from about 40° C. to about 48° C., from about 40° C. to about 50° C., from about 40° C. to about 52° C., from about 40° C. to about 55° C., from about 40° C. to about 58° C., from about 40° C. to about 60° C., from about 40° C. to about 65° C., from about 42° C. to about 45° C., from about 42° C. to about 48° C., from about 42° C. to about 50° C., from about 42° C. to about 52° C., from about 42° C. to about 55° C., from about 42° C. to about 58° C., from about 42° C. to about 60° C., from about 42° C. to about 65° C., from about 45° C. to about 48° C., from about 45° C. to about 50° C., from about 45° C. to about 52° C., from about 45° C. to about 55° C., from about 45° C. to about 58° C., from about 45° C. to about 60° C., from about 45° C. to about 65° C., from about 48° C. to about 50° C., from about 48° C. to about 52° C., from about 48° C. to about 55° C., from about 48° C. to about 58° C., from about 48° C. to about 60° C., from about 48° C. to about 65° C., from about 50° C. to about 52° C., from about 50° C. to about 55° C., from about 50° C. to about 58° C., from about 50° C. to about 60° C., from about 50° C. to about 65° C., from about 52° C. to about 55° C., from about 52° C. to about 58° C., from about 52° C. to about 60° C., from about 52° C. to about 65° C., from about 55° C. to about 58° C., from about 55° C. to about 60° C., from about 55° C. to about 65° C., from about 55° C. to about 70° C., from about 58° C. to about 60° C., from about 58° C. to about 65° C., from about 58° C. to about 70° C. An elevated temperature may be within a temperature range from about 37° C. to about 75° C., from about 40° C. to about 75° C., from about 45° C. to about 75° C., from about 50° C. to about 75° C., from about 51° C. to about 75° C., from about 52° C. to about 75° C., from about 53° C. to about 75° C., from about 54° C. to about 75° C., from about 55° C. to about 75° C. In other embodiments, the elevated temperature may be within the range of about 50° C. to about 70° C., from about 51° C. to about 70° C., from about 52° C. to about 70° C., from about 53° C. to about 70° C., from about 54° C. to about 70° C., from about 55° C. to about 70° C., from about 56° C. to about 65° C., from about 56° C. to about 64° C. or about 56° C. to about 62° C. In other embodiments, the elevated temperature may be within the range of about 46° C. to about 60° C., from about 47° C. to about 60° C., from about 49° C. to about 60° C., from about 51° C. to about 60° C., from about 53° C. to about 60° C., from about 54° C. to about 60° C., or from about 60° C. to about 85° C.

Such methods may include the use of one or more (e.g., one, two, three, four, five, ten, twelve, fifteen, etc.) DNA polymerases as part of the process of making the one or more double-stranded nucleic acid molecules. Such DNA polymerases are preferably thermostable DNA polymerases and most preferably the nucleic acid synthesis accomplished with such DNA polymerases is conducted at elevated temperatures, i.e., greater than 37° C. The invention also concerns compositions useful for making such double-stranded nucleic acid molecules. Such compositions comprise one or more inhibitors of the invention, one or more reverse transcriptases, and optionally one or more DNA polymerases, one or more suitable buffers or buffering salts, one or more primers, and/or one or more nucleotides. The invention further includes nucleic acid molecules prepared by the above methods and reaction mixtures used in and formed by such methods.

The invention also relates to methods for amplifying a nucleic acid molecule. Such amplification methods comprise mixing the double-stranded nucleic acid molecule or molecules produced as described above with one or more DNA polymerases (preferably thermostable DNA polymerases) and incubating the mixture under conditions sufficient to amplify the double-stranded nucleic acid molecule.

The invention further concerns a method for amplifying a nucleic acid molecule, the method comprising (a) mixing one or more (e.g., one, two, three, four, five, ten, twelve, fifteen, twenty, etc.) nucleic acid templates (preferably one or more RNA or mRNA templates and more preferably a population of mRNA templates), with one or more inhibitors of the invention, one or more reverse transcriptases or polypeptides having reverse transcriptase activity and one or more DNA polymerases and (b) incubating the mixture under conditions sufficient to amplify nucleic acid molecules complementary to all or a portion of the one or more templates. Such conditions may include incubation with one or more primers (such as oligo dT primers), one or more buffers or buffering salts, and/or one or more nucleotides. Preferably, the nucleic acid synthesis method of the invention is conducted under condition sufficient to inhibit, prevent, reduce, substantially reduce or eliminate degradation of the nucleic acid template or templates prior to and/or during nucleic acid synthesis. In some embodiments, the incubation of step (b) is performed at an elevated temperature. In a preferred aspect, the incubation temperature is determined by the temperature optimum of the enzyme(s) used in nucleic acid synthesis and such incubation temperature is preferably at the optimum or within 5° C. above or below the optimum. In specific embodiments, the elevated temperature may be from about 40° C. or greater, from about 45° C. or greater, from about 50° C. or greater, from about 51° C. or greater, from about 52° C. or greater, from about 53° C. or greater, from about 54° C. or greater, from about 55° C. or greater, from about 56° C. or greater, from about 57° C. or greater, from about 58° C. or greater, from about 59° C. or greater, from about 60° C. or greater, from about 61° C. or greater, from about 62° C. or greater, from about 63° C. or greater, from about 64° C. or greater, from about 65° C. or greater, from about 66° C. or greater, from about 67° C. or greater, from about 68° C. or greater, from about 69° C. or greater, from about 70° C. or greater, from about 71° C. or greater, from about 72° C. or greater, from about 73° C. or greater, from about 74° C. or greater, from about 75° C. or greater, from about 76° C. or greater, from about 77° C. or greater, from about 78° C. or greater, or from about 80° C. or greater. An elevated temperature may be within a temperature range of from about 40° C. to about 45° C., from about 40° C. to about 48° C., from about 40° C. to about 50° C., from about 40° C. to about 52° C., from about 40° C. to about 55° C., from about 40° C. to about 58° C., from about 40° C. to about 60° C., from about 40° C. to about 65° C., from about 42° C. to about 45° C., from about 42° C. to about 48° C., from about 42° C. to about 50° C., from about 42° C. to about 52° C., from about 42° C. to about 55° C., from about 42° C. to about 58° C., from about 42° C. to about 60° C., from about 42° C. to about 65° C., from about 45° C. to about 48° C., from about 45° C. to about 50° C., from about 45° C. to about 52° C.,. from about 45° C. to about 55° C., from about 45° C. to about 58° C., from about 45° C. to about 60° C., from about 45° C. to about 65° C., from about 48° C. to about 50° C., from about 48° C. to about 52° C., from about 48° C. to, about 55° C., from about 48° C. to about 58° C., from about 48° C. to about 60° C., from about 48° C. to about 65° C., from about 50° C. to about 52° C., from about 50° C. to about 55° C., from about 50° C. to about 58° C., from about 50° C. to about 60° C., from about 50° C. to about 65° C., from about 52° C. to about 55° C., from about 52° C. to about 58° C., from about 52° C. to about 60° C., from about 52° C. to about 65° C., from about 55° C. to about 58° C., from about 55° C. to about 60° C., from about 55° C. to about 65° C., from about 55° C. to about 70° C., from about 58° C. to about 60° C., from about 58° C. to about 65° C., from about 58° C. to about 70° C. An elevated temperature may be within a temperature range from about 37° C. to about 75° C., from about 40° C to about 75° C., from about 45° C. to about 75° C., from about 50° C. to about 75° C., from about 51° C. to about 75° C.,. from about 52° C. to about 75° C., from about 53° C. to about 75° C., from about 54° C. to about 75° C., from about 55° C. to about 75° C. In other embodiments, the elevated temperature may be within the range of about 50° C. to about 70° C., from about 51° C. to about 70° C., from about 52° C. to about 70° C., from about 53° C. to about 70° C., from about 54° C. to about 70° C., from about 55° C. to about 70° C., from about 56° C. to about 65° C., from about 56° C. to about 64° C. or about 56° C. to about 62° C. In other embodiments, the elevated temperature may be within the range of about 46° C. to about 60° C., from about 47° C. to about 60° C., from about 49° C. to about 60° C., from about 51° C. to about 60° C., from about 53° C. to about 60° C., from about 54° C. to about 60° C., or from about 60° C. to about 85° C.

Preferably, the reverse transcriptases used in the invention (1) are reduced or substantially reduced or lacking in RNase H activity, (2) are reduced or substantially reduced or lacking in TdT activity, and/or (3) exhibit increased fidelity. Preferably, the DNA polymerases used in the invention comprise a first DNA polymerase having 3′ exonuclease activity and a second DNA polymerase having substantially reduced 3′ exonuclease activity.

The invention further includes nucleic acid molecules prepared by the above methods and reaction mixtures or compositions used in and formed by such methods. Such compositions or reaction mixtures may comprise one or more components selected from the group consisting of one or more inhibitors, one or more reverse transcriptases, one or more DNA polymerases, one or more nucleotides, one or more suitable buffers or buffering salts (useful in nucleic acid synthesis or amplification) and one or more oligonucleotide primers. The compositions or reaction mixtures of the invention may further include one or more nucleic acid templates and or one or more nucleic acid molecules prepared by the methods of the invention.

The invention is also directed to nucleic acid molecules (particularly single- or double-stranded cDNA molecules) or amplified nucleic acid molecules produced according to the methods of the invention and to vectors (particularly expression vectors) comprising these nucleic acid molecules or amplified nucleic acid molecules.

The invention is further directed to recombinant host cells comprising the above-described nucleic acid molecules, amplified nucleic acid molecules or vectors. Examples of such host cells include bacterial cells, yeast cells, plant cells and animal cells (including insect cells and mammalian cells).

The invention is also directed to kits for use in methods of the invention. Such kits can be used for making, sequencing or amplifying nucleic acid molecules (single- or double-stranded), preferably at the elevated temperatures described herein. The kits of the invention may comprise a carrier, such as a box or carton, having in close confinement therein one or more (e.g., one, two, three, four, five, ten, twelve, fifteen, etc.) containers, such as vials, tubes, bottles and the like. In the kits of the invention, a first container contains one or more inhibitors of the present invention. The kits of the invention may also comprise reverse transcriptase enzymes or polypeptides having reverse transcriptase activity. The kits of the invention may also comprise one or more DNA polymerases (preferably thermostable DNA polymerases), one or more suitable buffers or buffering salts for nucleic acid synthesis, one or more host cells (preferably chemically competent or electrocompetent host cells such as E. coli), one or more nucleotides and one or more oligonucleotide primers. Any number of components of a kit of the invention may be combined into the same container. In a preferred aspect, the inhibitor of the invention is present in a container with one or more buffering salts or buffers and is preferably present in concentrated from such as 5 times, 10 times, 25 times, 50 times, 75 times, 100 times the working concentration of the inhibitor for use in the invention. Such concentrated solution may then be diluted to the appropriate working concentration for use according to the invention. Alternatively, the components of the kit may be divided into separate containers (e.g., one container for each enzyme and/or component). The kits of the invention also may comprise instructions or protocols for carrying out the methods of the invention. In preferred kits of the invention, the reverse transcriptases are reduced or substantially reduced or lacking in RNase H activity, and are most preferably selected from the group consisting of M-MLV RNase H minus reverse transcriptase, RSV RNase H minus reverse transcriptase, AMV RNase H minus reverse transcriptase, RAV RNase H minus reverse transcriptase, MAV RNase H minus reverse transcriptase and HIV RNase H minus reverse transcriptase. In other preferred kits of the invention, the reverse transcriptases are reduced or substantially reduced or lacking in TdT activity, and/or exhibit increased fidelity. In additional preferred kits of the invention, the enzymes (reverse transcriptases and/or DNA polymerases) in the containers are present at working concentrations. Thus, the invention is further directed to kits for use in reverse transcription, amplification or sequencing of a nucleic acid molecule, the kit comprising one or more inhibitors of the invention.

As indicated above, kits of the invention may contain any number of various components for practicing methods of the invention. One example of such a component is instructions for performing methods of the invention. As one skilled in the art would recognize, the full text of these instructions need not be included with the kit. One example of a situation in which kits of the invention would not contain such full length instructions is where directions are provided which inform individuals using the kits where to obtain instructions for using the kit. Thus, instructions for performing methods of the invention may be obtained from internet web pages, separately sold or distributed in manuals or other product literature, etc. The invention thus includes kits which direct kit users to locations where they can find instructions which are not directly packaged and/or distributed with the kits. These instructions may be in any form including, but not limited to, electronic or printed forms.

The invention thus also provides, in part, kits for performing methods using inhibitors of the invention. In specific embodiments, kits of the invention contain instructions for performing methods for amplifying and/or synthesizing nucleic acid molecules. These methods will often involve reacting RNA molecules with one or more inhibitors of the invention.

The invention is also directed to nucleic acid molecules produced by the above-described methods which may be full-length cDNA molecules, to vectors (particularly expression vectors) comprising these nucleic acid molecules and to host cells comprising these vectors and nucleic acid molecules.

Other preferred embodiments of the present invention will be apparent to one of ordinary skill in light of the following drawings and description of the invention, and of the claims.

### BRIEF DESCRIPTION OF THE FIGURES

FIG. 1. represents the half-lives of 7.5-Kb cRNA at various temperatures. RNA (7.5 Kb) was incubated at the temperatures and for the times indicated under reaction conditions optimal for M-MLV H−RT. The fraction relative to zero time of 7.5-Kb cRNA remaining intact at each time point was determined by agarose gel electrophoresis.

FIG. 2. represents the rate of loss of intact 7.5-Kb cRNA at various temperatures. The two initial data points at each temperature in FIG. 1 were re-plotted to determine the initial rate of 7.5-Kb cRNA breakdown. The data at 40 and 50° C. are not shown since the initial time points did not fall within the time scale shown.

FIG. 3. represents an EtBr-stained agarose gel showing the effect on 7.5-Kb RNA breakdown of EDTA and excess dNTPs with time at 60° C. Mixtures were incubated under conditions optimal for M-MLV H-RT with no additions (left), with EDTA added to 50 mM (center), or with additional dNTPs added to 4 mM total (right).

### DETAILED DESCRIPTION OF THE INVENTION

In order to provide a clearer and consistent understanding of the specifications and claims, including the scope to be given such terms, the following definitions are provided.

Inhibitor

As herein used, the term “inhibitor” refers to any compound or molecule or composition (or combination of the same or different molecules, compounds or compositions) that inhibits, prevents, substantially reduces or eliminates degradation or cleavage of RNA (and particularly poly A RNA or mRNA). In a preferred aspect, such inhibitors function or are used during nucleic acid synthesis (particularly during cDNA synthesis using one or more or a population of RNA molecules). In one aspect, the inhibitor of the invention does not adversely affect or substantially adversely affect nucleic acid synthesis. In one aspect, an inhibitor of the invention is not an enzyme that degrades or destroys a ribonuclease (RNase) such as RNase A, RNase H, RNase I, RNase III, or RNAase TI. In another aspect, the inhibitor of the invention is not a ribonuclease inhibitor such as RNaseOUT™ (Invitrogen Corporation), ANTI-RNase (Ambion), SUPERase.In™ (Ambion), or RNase Inhibitor (Roche). In another aspect, the inhibitor of the invention is not a protease. In another aspect, the inhibitor is not a protein or polyamino acid greater than 20 amino acids in length. In yet another aspect, the inhibitor is not an enzyme. In a preferred aspect, the inhibitor of the invention is not EDTA, EGTA and/or citrate. In another aspect, Na pyrophosphate is not a preferred inhibitor. In another aspect, the inhibitor is not a chelator which adversely affects or substantially adversely affects nucleic acid synthesis. In one aspect, the inhibitor is a chemical compound. In one embodiment, the inhibitor is one or more (or a combination of) compounds or molecules or compositions that are negatively charged or have a net negative charge. In one aspect, the inhibitor of the invention binds (covalently, non-covalently, reversibly and/or irreversibly) to one or more degradation components. Examples of inhibitors of the invention include but are not limited to one or more nucleotides, one or more anions, one or more basic salts, one or more chelators, one or more amino acids, one or more polyamino acids 20 amino acids in length or shorter, or combinations thereof.

Degradation Component

The term “degradation component” refers to a compound, molecule, or protein, or combinations thereof, which is capable of RNA degradation or cleavage preferably during synthesis of nucleic acids. Such degradation component of the invention preferably is positively charged or has a net positive charge. Examples of degradation components include but are not limited to, metal ions, cations, cationic salts, or any other compound or molecules having a net positive charge.

Nucleotide

As herein used, the term “nucleotide” refers to a base-sugar-phosphate combination. Nucleotides include monomeric units of a nucleic acid sequence (DNA and RNA). The term nucleotide includes ribo- and deoxy-nucleoside monophosphates, diphosphates, and /or triphosphates and derivatives thereof. The term includes ribonucleoside triphosphate ATP, UTP, CTP, GTP and deoxyribonucleoside triphosphates such as dATP, dCTP, dITP, dUTP, dTTP, or derivatives thereof. Such derivatives include, for example, [αS]dATP, 7-deaza-dGTP and 7-deaza-dATP, and nucleotide derivatives that confer nuclease resistance on the nucleic acid molecule containing them. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. The term nucleotide as used herein also refers to deoxyribonucleoside monophosphates and ribonucleoside monophosphates such as dAMP, dGMP, dCMP, dTMP, dUMP, AMP, GMP, CMP, UMP, and their derivatives. Nucleotide also includes ribonucleoside diphosphates and deoxyribonucleoside diphosphates such as dADP, dGDP, dCDP, dTDP, dUDP, ADP, GDP, CDP, UDP and their derivatives. The term nucleotide also refers to nucleotides with oxidized, alkylated or methylated bases, for example thymine glycol, 8-oxoguanine, 4,6-diamino-5-formamidopyrimidine, urea, 3-methyladenine, 7-methylguanine, or 6-methylguanine. According to the present invention, a nucleotide may be unlabeled or detectably labeled by well-known techniques. Detectable labels include, for example, radioactive isotopes, fluorescent labels, chemiluminescent labels, bioluminescent labels and enzyme labels.

Amino Acid

The term “amino acid” as used herein refers generally to a molecule of the formula NH₂—CHR—COOH, wherein R is a side chain or residue, which may or may not occur naturally. The terms “natural amino acid” and “naturally-occurring amino acid” refer to those 20 amino acids which are the normal constituents of proteins, e.g., D amino acids such as Ala, Asp, Cys, Glu, Phe, Gly, His, Ile, Lys, Leu, Met, Asn, Pro, Gln, Arg, Ser, Thr, Val, Trp, and Tyr. In addition, the term “amino acid” also includes other non-naturally occurring amino acids besides the D-amino acids, which are functional equivalents of the naturally-occurring amino acids. Such non-natural amino acids also include L-forms of the 20 amino acids normally found in proteins. Such non-naturally-occurring amino acids also include, for example, norleucine (“Nle”), norvaline (“Nva”), beta.-Alanine, L- or D-naphthalanine, ornithine (“Orn”), and homoarginine (homoArg). Other (non-natural) amino acids which may be used include homoserine, phenylglycine, taurine, iodotyrosine, and others well known in the peptide art, such as those described in M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill., 1984, both of which are incorporated herein by reference. Amino acids and amino acid analogs can be purchased commercially (Sigma Chemical Co.; Advanced Chemtech) or synthesized using methods known in the art. Preferably, the side chain (R) of an amino acid will contain 1-12 carbon atoms, 0-4 nitrogen atoms, 0-2 sulfurs, 0-4 oxygens, and 0-4 halogen atoms.

Anion

The term “anion,” as herein used, refers generally to compounds or molecules having a net negative charge or salts thereof. Examples of such a compound or molecule include but are not limited to Cl⁻, Br⁻, I, ClO₄⁻, BrO₄⁻, ClO₃⁻, NO₃⁻, F⁻, I⁻, ClO₁⁻, ClO₂⁻, ClO⁻, BrO₃⁻, IO₃⁻, OH⁻, SH⁻, S₂²⁻, HSO₄⁻, SO₃²⁻, SO₄²⁻, SeO₃²⁻, SeO₄²⁻, NO₂⁻, NO₃⁻, N₃⁻, PO₄³⁻, CO₃²⁻, HCO₃⁻, CN⁻, BF₄⁻, MnO₄⁻, CrO₄⁻, and Cr₂O₇²⁻.

Chelator

As herein used, the term “chelator” refers to a compound or molecule that binds to a metal ion and may include salts of such compounds or molecules. In many instances, when present in suitable concentrations, chelators will protect RNA from degradation caused by a metal ion during reverse transcription. The term chelator includes, but is not limited to, Na pyrophosphate, EDTAs, EGTAs, sodium citrates (citric acids), picolinate, salicyclic acids (and their salts), phthalic acids, 2,4-pentanediones, histidines, histidinol dihydrochlorides, 8-hydroxylquinolines, 8-hydroxyquinoline citrates, and o-hydroxylquinones, or derivatives of benzoic acid or hydroxamic acid.

Primer

As used herein, the term “primer” refers to a single-stranded oligonucleotide that is extended by covalent bonding of nucleotide monomers during amplification or polymerization of a nucleic acid molecule.

Template

The term “template” as herein used refers to double-stranded or single-stranded nucleic acid molecules that are to be amplified, synthesized or sequenced. In the case of double-stranded molecules, denaturation of its strands to form a first and a second strand is preferably performed before these molecules may be amplified, synthesized, or sequenced, or the double stranded molecule may be used directly as a template. For single-stranded templates, a primer, complementary to a portion of the template is hybridized under appropriate conditions and one or more polymerases and/or reverse transcriptases may then synthesize or amplify one or more a nucleic acid molecules complementary to all or a portion of said template. The newly synthesized molecules, according to the invention, may be equal or shorter in length than the original template. Additionally, the synthesized or amplified nucleic acid molecules may be complementary to all or a portion of the template and/or may have mismatched bases or different bases compared to the template.

Incorporating

The term “incorporating” as used herein means becoming part of a DNA and/or RNA molecule or primer.

Amplification

As used herein “amplification” refers to any in vitro method for increasing the number of copies of a nucleotide sequence with use of a polymerase. Nucleic acid amplification results in the incorporation of nucleotides into a DNA and/or RNA molecule or primer thereby forming a new molecule complementary to all or a portion of the template. The formed nucleic acid molecule and its template can be used as templates to synthesize additional nucleic acid molecules. As used herein, one amplification reaction may consist of many rounds of replication. DNA amplification reactions include, for example, polymerase chain reactions (PCR). One PCR reaction may consist of 5 to 100 “cycles” of denaturation and synthesis of a DNA molecule.

Host

As used herein “host” means any prokaryotic or eukaryotic organism that is the recipient of a replicable expression vector, cloning vector or any nucleic acid molecule. The nucleic acid molecule may contain, but is not limited to, a structural gene, a promoter and/or an origin of replication.

Hybridization

The terms “hybridization” and “hybridizing” refers to base pairing of two complementary single-stranded nucleic acid molecules (RNA and/or DNA) to give a double-stranded molecule. As used herein, two nucleic acid molecules may be hybridized, although the base pairing is not complementary. Accordingly, mismatched bases do not prevent hybridization of two nucleic acid molecules provided that appropriate conditions, well known in the art, are used.

Vector

As herein used, “vector” is a plasmid, cosmid, phagemid or phage DNA or other DNA molecule which is able to replicate autonomously in a host cell, and which is characterized by one or a small number of restriction endonuclease recognition sites at which such DNA sequences may be cut in a determinable fashion without loss of an essential biological function of the vector, and into which DNA may be inserted in order to bring about its replication and cloning. The vector may further contain a marker suitable fore use in the identification of cells transformed with the vector. Markers, for example, include but are not limited to tetracycline resistance or amnpicillin resistance.

Oligonucleotide

“Oligonucleotide” refers to a synthetic or natural molecule comprising a covalently linked sequence of nucleotides which are joined by a phosphodiester bond between the 3′ position of the deoxyribose or ribose of one nucleotide and the 5′ position of the deoxyribose or ribose of the adjacent nucleotide.

Library

As used herein, the term “library” or “nucleic acid library” means a set of nucleic acid molecules (circular or linear) representative of all or a significant portion of the DNA content of an organism (a “genomic library”), or a set of nucleic acid molecules representative of all or a significant portion of the expressed genes (a “cDNA library”) in a cell, tissue, organ, or organism. Such libraries may or may not be contained in one or more vectors.

Reduced

As herein used, the term “reduced” refers to a reduction in the amount or level of degradation of RNA (particularly mRNA molecules or poly A⁺ RNA molecules, or a population of RNA molecules). Such reduction is preferably a reduction of greater than 0%, more preferably greater than 1%, still more preferably greater than 5%, still more preferably greater than 10%, still more preferably greater than 20%, still more preferably greater than 30%, still more preferably greater than 40%, still more preferably greater than 45%, still more preferably greater than 49% compared to the level of degradation prior to treatment or without treatment in accordance with the invention. In an aspect, the amount or level of degradation is compared with and without the addition of an inhibitor and preferably such comparison is made at one or any number of times during a cDNA synthesis reaction. In another aspect, the degradation level obtained using prior art cDNA synthesis reaction conditions is compared to the degradation level obtained using conditions of the invention.

Substantially Reduced

As herein used, the term “substantially reduced” refers to a reduction in the amount or level of degradation of RNA (particularly mRNA molecules or poly A⁺ RNA molecules, or a population of RNA molecules). Such reduction is preferably a reduction of greater than 50%, more preferably greater than 60%, still more preferably greater than 70%, still more preferably greater than 80%, still more preferably greater than 95% compared to the level of degradation incurred prior to treatment in accordance with the invention. In an aspect, the amount or level of degradation is compared with and without the addition of an inhibitor and preferably such comparison is made at one or any number of times during a cDNA synthesis reaction. In another aspect, the degradation level obtained using prior art cDNA synthesis reaction conditions is compared to the degradation level obtained using conditions of the invention.

Eliminate

As herein used, the term “eliminate” means that the level or amount of RNA degradation during nucleic acid synthesis is diminished completely or is undetectable.

Does Not Substantially Adversely Affect Nucleic Acid Synthesis

As used herein this phrase means that the amount of nucleic acid produced during nucleic acid synthesis with inhibitor is not reduced by more than 40%, is not reduced by more than 35%, is not reduced by more than 30%, is not reduced by more than 25%, is not reduced by more than 20%, is not reduced by more than 15%, is not reduced by more than 10%, is not reduced by more than 5%, is not reduced by more than 4%, is not reduced by more than 3%, is not reduced by more than 2%, or is not reduced by more than 1% compared to nucleic acid synthesis without inhibitor. The amount of nucleic acid produced using prior art cDNA synthesis reaction conditions may be compared to the amount of nucleic acid produced using the conditions of the invention, to determine the percent reduction (if any) of nucleic acid synthesis.

Full Length

As used herein, full length refers to a product molecule, e.g., a cDNA molecule, that is the same length or substantially the same length as the template molecule, e.g., an mRNA molecule, from which it is produced. The term “substantially the same length as the template” means that the molecule produced (e.g. cDNA) is at least 90% or at least 95% or more the base length of the template molecule (e.g. mRNA). Template molecules may be from about 100 bases to about 50 kb in length, from about 200 bases to about 50 kb in length, from about 300 bases to about 50 kb in length, from about 400 bases to about 50 kb in length, from about 500 bases to about 50 kb in length, from about 600 bases to about 50 kb in length, from about 700 bases to about 50 kb in length, from about 800 bases to about 50 kb in length, from about 900 bases to about 50 kb in length, and from about 1 kb to about 50 kb in length. In some embodiments, template molecules may be from about 500 bases to about 10 kb in length, from about 600 bases to about 10 kb in length, from about 700 bases to about 10 kb in length, from about 800 bases to about 10 kb in length, from about 900 bases to about 10 kb in length, from about 1000 bases to about 10 kb in length, from about 1100 bases to about 10 kb in length, and/or from about 1200 bases to about 10 kb in length. In some embodiments, template molecules may be from about 250 bases to about 5 kb in length, from about 300 bases to about 5 kb in length, and from about 350 bases to about 5 kb in length, from about 400 bases to about 5 kb in length, from about 450 bases to about 5 kb in length, from about 500 bases to about 5 kb in length, from about 550 bases to about 5 kb in length, from about 600 bases to about 5 kb in length, from about 650 bases to about 5 kb in length, from about 700 bases to about 5 kb in length, from about 750 bases to about 5 kb in length, from about 800 bases to about 5 kb in length, and from about 850 bases to about 5 kb in length.

### Overview

The present invention provides compositions and methods useful in overcoming RNA degradation often observed during reverse transcription of nucleic acid molecules. Thus, the invention facilitates the production of full-length cDNA molecules by using an inhibitor to prevent, reduce, substantially reduce, or eliminate RNA degradation during nucleic acid synthesis.

In general, the invention provides compositions for use in reverse transcription of a nucleic acid molecule comprising one or more (e.g., one, two, three, four, five, ten, twelve, fifteen, twenty, thirty, etc.) inhibitors that are capable of inhibiting degradation of RNA. The inhibitor preferably interacts or binds (reversibly or irreversibly) with one or more degradation components present in the nucleic acid synthesis reaction (e.g. cDNA synthesis reaction) thus protecting the RNA from degradation or hydrolysis typically caused by the degradation component.

The invention generally relates to methods to protect one or more RNA molecules from hydrolysis or degradation comprising contacting or mixing one or more inhibitors of the invention with one or more RNA molecules and incubating under conditions sufficient to prevent, reduce, substantially reduce or eliminate degradation or hydrolysis of said RNA molecules. The protected RNA molecules may be used as templates for nucleic acid synthesis (e.g. cDNA synthesis). The invention therefore may provide for more efficient synthesis or amplification of nucleic acid molecules and preferably allows for an increase in the total amount of cDNA produced and/or allows for an increase in the amount of full-length cDNA produced compared to cDNA synthesis without an inhibitor of the invention or compared to prior art cDNA synthesis reaction conditions.

The invention is also directed to methods for reverse transcription of one or more (e.g., one, two, three, four, five, ten, twelve, fifteen, twenty, etc.) nucleic acid molecules comprising mixing one or more (e.g., one, two, three, four, five, ten, twelve, fifteen, twenty, etc.) inhibitors of the invention with one or more (e.g., one, two, three, four, five, ten, twelve, fifteen, twenty, etc.) nucleic acid templates, which is preferably RNA or messenger RNA (niRNA) and more preferably a population of mRNA molecules, and one or more reverse transcriptases or polypeptides having reverse transcription activity and incubating the mixture under conditions sufficient to make a nucleic acid molecule or molecules complementary to all or a portion of the one or more templates. To make the nucleic acid molecule or molecules complementary to the one or more templates, a primer (e.g., an oligo(dT) primer) and one or more nucleotides are preferably used for nucleic acid synthesis in the 5′ to 3′ direction. Nucleic acid molecules suitable for reverse transcription according to this aspect of the invention include any nucleic acid molecule, particularly those derived from a prokaryotic or eukaryotic cell. Such cells may include normal cells, diseased cells, transformed cells, established cells, progenitor cells, precursor cells, fetal cells, embryonic cells, bacterial cells, yeast cells, animal cells (including human cells), avian cells, plant cells and the like, or tissue isolated from a plant or an animal (e.g., human, cow, pig, mouse, sheep, horse, monkey, canine, feline, rat, rabbit, bird, fish, insect, etc.). Such nucleic acid molecules may also be isolated from viruses.

The invention further provides methods for amplifying or sequencing a nucleic acid molecule comprising contacting the nucleic acid molecule with one or more inhibitors of the present invention. Preferred such methods comprise one or more polymerase chain reactions (PCRs).

### Sources of Inhibitor

Inhibitors for use in the compositions, methods, and kits of the invention include nucleotides, anions, and chelators. The preferred inhibitors of the invention may vary depending on the DNA polymerase, reverse transcriptase, and/or other components used in the composition or reaction mixture of the invention. One of ordinary skill can, without undue experimentation, test various inhibitors for use in the desired system and select the optimal combination of inhibitor(s) and/or other reaction components used in the a particular system of interest.

Examples of nucleotides suitable for use in the present compositions include, but are not limited to, dUTP, dATP, dTTP, dCTP, dGTP, dITP, 7-deaza-dGTP, α-thio-dATP, α-thio-dTTP, α-thio-dGTP, α-thio-dCTP, ddUTP, ddATP, ddTTP, ddCTP, ddGTP, ddITP, 7-deaza-ddGTP, α-thio-ddATP, α-thio-ddTTP, α-thio-ddGTP, α-thio-ddCTP or derivatives thereof, all of which are available commercially from sources including Invitrogen Corporation (Carlsbad, Calif.), New England BioLabs (Beverly, Mass.) and Sigma Chemical Company (Saint Louis, Mo.). The nucleotides may be unlabeled, or they may be detectably labeled by coupling them by methods known in the art with radioisotopes (e.g., ³H, ¹⁴C, ³²P or ³⁵S), vitamins (e.g., biotin), fluorescent moieties (e.g., fluorescein, rhodamine, Texas Red, or phycoerythrin), chemiluminescent labels (e.g., using the PHOTO-GENE™ or ACES™ chemiluminescence systems, available commercially from Invitrogen Corporation (Carlsbad, Calif.)), dioxigenin and the like. Labeled nucleotides may also be obtained commercially, for example from Invitrogen Corporation (Carlsbad, Calif.) or Sigma Chemical Company (Saint Louis, Missouri).

Examples of anions include but are not limited to Cl⁻, Br⁻, I, ClO₄⁻, BrO₄⁻, ClO₃⁻, NO₃⁻, F⁻, I⁻, ClO₁⁻, ClO₂⁻, ClO⁻, BrO₃⁻, IO₃⁻, OH⁻, SH⁻, S₂²⁻, HSO₄⁻, SO₃²⁻, SO₄²⁻, SeO₃²⁻, SeO₄²⁻, NO₂⁻, NO₃⁻, N₃⁻, PO₄³⁻, CO₃²⁻, HCO₃⁻, CN⁻, BF₄⁻, MnO₄⁻, CrO₄⁻, and Cr₂O₇²⁻, all of which are commercially available from Sigma Chemical Company (Saint Louis, Mo.).

The term chelator includes, but is not limited to, Na pyrophosphate, EDTAs, EGTAs, sodium citrates (citric acids), 2,4-pentanediones, histidines, histidinol dihydrochlorides, or derivatives of benzoic acid or hydroxamic acid, all which are available from Sigma Chemical Company (Saint Louis, Mo.). Other chelators which may be used are picolinate, salicyclic acids (and their salts) phthalic acids, 8-hydroxylquinolines, 8-hydroxyquinoline citrates, and o-hydroxylquinones, all available from VWR International.

### Sources of Enzyme

Enzymes for use in the compositions, methods and kits of the invention include any enzyme having reverse transcriptase activity. Such enzymes include, but are not limited to, retroviral reverse transcriptase, retrotransposon reverse transcriptase, hepatitis B reverse transcriptase, cauliflower mosaic virus reverse transcriptase (CMV-RT), bacterial reverse transcriptase, Rausher Leukemia Virus (RLV-RT), Mouse Mammary Tumor Virus (MMTV-RT), Tobacco Mosaic Virus (TMV-RT), Human Foamy Virus HMV-RT), Tth DNA polymerase, Taq DNA polymerase (Saiki, R. K., et al., Science 239:487-491 (1988); U.S. Pat. Nos. 4,889,818 and 4,965,188), Tne DNA polymerase (PCT Publication No. WO 96/10640), Tma DNA polymerase (U.S. Pat. No. 5,374,553) and mutants, fragments, variants or derivatives thereof (see, e.g., commonly owned U.S. Pat. Nos. 5,948,614 and 6,015,668, which are incorporated by reference herein in their entireties). Preferably, reverse transcriptases for use in the invention include retroviral reverse transcriptases such as M-MLV reverse transcriptase, AMV reverse transcriptase, HIV reverse transcriptases, RSV reverse transcriptase, RAV reverse transcriptase, MAV reverse transcriptase, and generally ASLV reverse transcriptases. Additional reverse transcriptases which may be used to prepare compositions of the invention include bacterial reverse transcriptases (e.g., Escherichia coli reverse transcriptase) (see, e.g., Mao et al., Biochem. Biophys. Res. Commun. 227:489-93 (1996)) and reverse transcriptases of Saccharomyces cerevisiae (e.g., reverse transcriptases of the Ty1 or Ty3 retrotransposons) (see, e.g., Cristofari et al., Jour. Biol. Chem. 274:36643-36648 (1999); Mules et al., Jour. Virol. 72:6490-6503 (1998)). As will be understood by one of ordinary skill in the art, modified reverse transcriptases or modified DNA polymerases may be obtained by recombinant or genetic engineering techniques that are routine and well-known in the art. Mutant reverse transcriptases or mutant DNA polymerases can, for example, be obtained by mutating the gene or genes encoding the reverse transcriptase or DNA polymerase of interest by site-directed or random mutagenesis. Such mutations may include point mutations, deletion mutations and insertional mutations. For example, one or more point mutations (e.g., substitution of one or more amino acids with one or more different amino acids) may be used to construct mutant reverse transcriptases or DNA polymerases for use in the invention.

Preferred enzymes for use in the invention include those that are reduced or substantially reduced or lacking in RNase H activity. Such enzymes that are reduced or substantially reduced or lacking in RNase H activity may be obtained by mutating, for example, the RNase H domain within the reverse transcriptase of interest, for example, by introducing one or more (e.g., one, two, three, four, five, ten, twelve, fifteen, twenty, thirty, etc.) point mutations, one or more (e.g., one, two, three, four, five, ten, twelve, fifteen, twenty, thirty, etc.) deletion mutations, and/or one or more (e.g., one, two, three, four, five, ten, twelve, fifteen, twenty, thirty, etc.) insertion mutations as described above.

By an enzyme “reduced in RNase H activity” is meant any detectable reduction (for example, 1% or greater) in RNase H activity compared to the corresponding wild-type or RNase H⁺ enzymes. By an enzyme “substantially reduced in RNase H activity” is meant that the enzyme has less than about 30%, less than about 25%, less than about 20%, more preferably less than about 15%, less than about 10%, less than about 7.5%, or less than about 5%, and most preferably less than about 5% or less than about 2%, of the RNase H activity of the corresponding wild-type or RNase H⁺ enzyme, such as wild-type Moloney Murine Leukemia Virus (M-MLV), Avian Myeloblastosis Virus (AMV) or Rous Sarcoma Virus (RSV) reverse transcriptases.

Reverse transcriptases having reduced or substantially reduced or lacking RNase H activity have been previously described (see U.S. Pat. No. 5,668,005, U.S. Pat. No. 6,063,608, and PCT Publication No. WO 98/47912). The RNase H activity of any enzyme may be determined by a variety of assays, such as those described, for example, in U.S. Pat. No. 5,244,797, in Kotewicz, M. L., et al., Nucl. Acids Res. 16:265 (1988), in Gerard, G. F., et al., FOCUS 14(5):91 (1992), and in U.S. Pat. No. 5,668,005, the disclosures of all of which are fully incorporated herein by reference. Enzymes “lacking” in RNase H activity shall mean the RNase H activity is undetectable by the gel assay and/or the solubilization assay described in US Pat. No. 5,668,005. Preferred enzymes for use in the invention include Superscript™, Superscript II™, Thermoscript™, Fluoroscript™, M-MLV Reverse Transcriptase, and AMV Reverse Transcriptase, all available from Invitrogen Corporation.

Particularly preferred enzymes for use in the invention include, but are not limited to, M-MLV RNase H minus reverse transcriptase, RSV RNase H minus reverse transcriptase, AMV RNase H minus reverse transcriptase, RAV RNase H minus reverse transcriptase, MAV RNase H minus reverse transcriptase and HWV RNase H minus reverse transcriptase. It will be understood by one of ordinary skill, however, that any enzyme capable of producing a DNA molecule from a ribonucleic acid molecule (i.e., having reverse transcriptase activity) that is reduced or substantially reduced in RNase H activity may be equivalently used in the compositions, methods and kits of the invention.

In additional aspects, thermostable reverse transcriptases are used in the invention, which retain at least about 50%, at least about 60%, at least about 70%, at least about 85%, at least about 95%, at least about 97%, at least about 98%, at least about 99%, at least about 100% of reverse transcriptase activity after heating to 50° C. for 5 minutes.

Enzymes for use in the invention also include modified or mutated reverse transcriptase (e.g., a modified or mutated retroviral reverse transcriptase) having a reverse transcriptase activity that has a half-life of greater than that of the corresponding un-modified or un-mutated reverse transcriptase at an elevated temperature, i.e., greater than 37° C. In some embodiments, the half-life of a reverse transcriptase of the present invention may be 5 minutes or greater and preferably 10 minutes or greater at 50° C. In some embodiments, the reverse transcriptases of the invention may have a half-life at 50° C. equal to or greater than about 25 minutes, preferably equal to or greater than about 50 minutes, more preferably equal to or greater than about 100 minutes, and most preferably, equal to or greater than about 200 minutes at 50° C. In some embodiments, the reverse transcriptases of the invention may have a half-life at 50° C. that is from about 10 minutes to about 200 minutes, from about 10 minutes to about 150 minutes, from about 10 minutes to about 100 minutes, from about 10 minutes to about 75 minutes, from about 10 minutes to about 50 minutes, from about 10 minutes to about 40 minutes, from about 10 minutes to about 30 minutes, or from about 10 minutes to about 20 minutes. Reverse transcriptases which exhibit increased thermostability are described in U.S. appl. Ser. No. 09/845,157, filed May 1, 2001 and PCT Publication No. WO 01/92500 (the entire disclosure of which is incorporated herein by reference)

Enzymes for use in the invention also include those in which terminal deoxynucleotidyl transferase (TdT) activity has been reduced, substantially reduced, or eliminated. Such enzymes that are reduced or substantially reduced in terminal deoxynucleotidyl transferase activity, or in which TdT activity has been eliminated, may be obtained by mutating, for example, amino acid residues within the reverse transcriptase of interest which are in close proximity or in contact with the template-primer, for example, by introducing one or more (e.g. one, two, three, four, five, ten, twelve, fifteen, twenty, thirty, etc.) point mutations, one or more deletion mutations, and/or one or more insertion mutations. Reverse transcriptases which exhibit decreased TdT activity are described in U.S. appl. Ser. No. 09/808,124, filed Mar. 15, 2001 (the entire disclosure of which is incorporated herein by reference), and include reverse transcriptases with one or more alterations at amino acid positions equivalent or corresponding to Y64, M289, F309, T197 and/or Y133 of M-MLV reverse transcriptase.

Enzymes for use in the invention also include those which exhibit increased fidelity. Fidelity refers to the accuracy of polymerization, or the ability of the reverse transcriptase to discriminate correct from incorrect substrates, (e.g., nucleotides) when synthesizing nucleic acid molecules which are complementary to a template. The higher the fidelity of a reverse transcriptase, the less the reverse transcriptase misincorporates nucleotides in the growing strand during nucleic acid synthesis; that is, an increase or enhancement in fidelity results in a more faithful reverse transcriptase having decreased error rate or decreased misincorporation rate.

A reverse transcriptase having increased/enhanced/higher fidelity is defined as a polymerase having any increase in fidelity, preferably about 1.2 to about 10,000 fold, about 1.5 to about 10,000 fold, about 2 to about 5,000 fold, or about 2 to about 2000 fold (preferably greater than about 5 fold, more preferably greater than about 10 fold, still more preferably greater than about 50 fold, still more preferably greater than about 100 fold, still more preferably greater than about 500 fold and most preferably greater than about 100 fold) reduction in the number of misincorporated nucleotides during synthesis of any given nucleic acid molecule of a given length compared to the control reverse transcriptase. Reverse transcriptases which exhibit increased fidelity are described in U.S. appl. Ser No. 60/189,454, filed Mar. 15, 2000, U.S. appl. Ser. No. 09/808,124, filed Mar. 15, 2001, U.S. appl. Ser. No. 60/056,263, filed Aug. 29, 1997, U.S. appl. Ser. No. 60/060,131, filed Sep. 26, 1997, U.S. appl. Ser. No. 60/085, 247, filed May 13, 1998, U.S. applicaton Ser. No. 09/141,522, filed Aug. 27, 1998, U.S. appl. Ser. No. 09/677, 574, filed Aug. 3, 2000; PCT Publication No. WO 00/204022; and PCT Publication No. WO 01/68895 (the entire disclosures of each of which are incorporated herein by reference).

Suitable host for cloning the reverse transcriptase genes and nucleic acid molecules of interest are prokaryotic hosts. One example of a prokaryotic host is E. coli. However, the desired reverse transcriptase genes and nucleic acid molecules of the present invention may be cloned in other prokaryotic hosts including, but not limited to, hosts in the genera Escherichia, Bacillus, Streptomyces, Pseudomonas, Salmonella, Serratia, and Proteus. Bacterial hosts of particular interest include E. coli DH10B, DH5, DH5α, Top 10 etc., which may be obtained from Invitrogen Corporation (Carlsbad, Calif.).

Eukaryotic hosts for cloning and expression of the reverse transcriptase of interest include yeast, fungal, and mammalian cells. Expression of the desired reverse transcriptase gene or other genes or partial genes in such eukaryotic cells may require the use of eukaryotic regulatory regions which include eukaryotic promoters. Cloning and expressing the reverse transcriptase gene or nucleic acid molecule in eukaryotic cells may be accomplished by well known techniques using well known eukaryotic vector systems.

### Sources of DNA Polymerase

A variety of DNA polymerases are useful in accordance with the present invention including thermostable and mesophilic DNA polymerases. In one aspect, preferred DNA polymerases are those that have reverse transcriptase activity (template reading in the 3′ to 5′ direction) and/or DNA polymerase activity (e.g. template reading in the 5′ to 3′ direction). Such polymerases for use in the invention include, but are not limited to, pol I type and pol III type DNA polymerases. Examples of thermostable DNA polymerase for use in the invention include Thermus thermophilus (Tth) DNA polymerase, Thermus aquaticus (Taq) DNA polymerase, Thermotoga neapolitana (Tne) DNA polymerase, Thermotoga maritima (Tma) DNA polymerase, Thermococcus litoralis (Tli or VENT™) DNA polymerase, Pyrococcus furiosis (Pfu) DNA polymerase, Pyrococcus species GB-D (DEEPVENT™) DNA polymerase, Pyrococcus woosii (Pwo) DNA polymerase, Bacillus sterothermophilus (Bst) DNA polymerase, Bacillus caldophilus (Bca) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME™) DNA polymerase, Methanobacterium thermoautotrophicum (Mth) DNA polymerase, Mycobacterium spp. DNA polymerase (Mtb, Mlep), and mutants, variants and derivatives thereof. Mesophilic polymerases include DNA polymerase I, T5 DNA polymerase, T7 DNA polymerase, Klenow fragment DNA polymerase, DNA polymerase II, and the like.

Preferred DNA polymerases are thermostable DNA polymerases such as Taq, Tne, Tma, Pfu, VENT™, DEEPVENT™, Tth and mutants, variants and derivatives thereof (U.S. Pat. No. 5,436,149; U.S. Pat. No. 5,512,462; PCT Publication No. WO 92/06188; PCT Publication No. WO 92/06200; PCT Publication No. WO 96/10640; Barnes, W. M., Gene 112:29-35 (1992); Lawyer, F. C., et al., PCR Meth. Appl. 2:275-287 (1993); Flaman, J.-M., et al, Nucl. Acids Res. 22(15):3259-3260 (1994)). Other DNA polymerases for use in the invention may be found in U.S. application Ser. No. 60/318,903, filed Sep. 14, 2001, and U.S. patent application U.S. 2002/0012970. For amplification of long nucleic acid molecules (e.g., nucleic acid molecules longer than about 3-5 Kb in length), at least two DNA polymerases (one substantially lacking 3′ exonuclease activity and the other having 3′ exonuclease activity) are typically used. See U.S. Pat. No. 5,436,149; U.S. Pat. No. 5,512,462; Barnes, W. M., Gene 112:29-35 (1992); PCT Publication No. WO 98/06736; and commonly owned, co-pending U.S. patent application Ser. No. 08/801,720, filed Feb. 14, 1997, the disclosures of all of which are incorporated herein in their entireties. Examples of DNA polymerases substantially lacking in 3′ exonuclease activity include, but are not limited to, Taq, Tne(exo⁻), Tma, Pfu(exo⁻), Pwo and Tth DNA polymerases, and mutants, variants and derivatives thereof. Nonlimiting examples of DNA polymerases having 3′ exonuclease activity include Pfu, DEEPVENT™ and Tli/VENT™ and mutants, variants and derivatives thereof.

### Production/Sources of cDNA Molecules

In accordance with the invention, cDNA molecules (single-stranded or double-stranded) may be prepared from a variety of nucleic acid template molecules. Preferred nucleic acid molecules for use in the present invention include single-stranded or double-stranded DNA and RNA molecules, as well as double-stranded DNA:RNA hybrids. More preferred nucleic acid molecules include messenger RNA (mRNA), poly A RNA, transfer RNA (tRNA) and ribosomal RNA (rRNA) molecules, although mRNA and poly A RNA molecules are the preferred templates according to the invention.

The nucleic acid molecules that are used to prepare cDNA molecules according to the methods of the present invention may be prepared synthetically according to standard organic chemical synthesis methods that will be familiar to one of ordinary skill. More preferably, the nucleic acid molecules may be obtained from natural sources, such as a variety of cells, tissues, organs or organisms. Cells that may be used as sources of nucleic acid molecules may be prokaryotic (bacterial cells, including but not limited to those of species of the genera Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, Xanthomonas and Streptomyces) or eukaryotic (including fungi (especially yeasts), plants, protozoans and other parasites, and animals including insects (particularly Drosophila spp. cells), nematodes (particularly Caenorhabditis elegans cells), and mammals (particularly human cells)).

Mammalian somatic cells that may be used as sources of nucleic acids include blood cells (reticulocytes and leukocytes), endothelial cells, epithelial cells, neuronal cells (from the central or peripheral nervous systems), muscle cells (including myocytes and myoblasts from skeletal, smooth or cardiac muscle), connective tissue cells (including fibroblasts, adipocytes, chondrocytes, chondroblasts, osteocytes and osteoblasts) and other stromal cells (e.g., macrophages, dendritic cells, Schwann cells). Mammalian germ cells (spermatocytes and oocytes) may also be used as sources of nucleic acids for use in the invention, as may the progenitors, precursors and stem cells that give rise to the above somatic and germ cells. Also suitable for use as nucleic acid sources are mammalian tissues or organs such as those derived from brain, kidney, liver, pancreas, blood, bone marrow, muscle, nervous, skin, genitourinary, circulatory, lymphoid, gastrointestinal and connective tissue sources, as well as those derived from a mammalian (including human) embryo or fetus.

Any of the above prokaryotic or eukaryotic cells, tissues and organs may be normal, diseased, transformed, established, progenitors, precursors, fetal or embryonic. Diseased cells may, for example, include those involved in infectious diseases (caused by bacteria, fungi or yeast, viruses (including AIDS, HIV, HTLV, herpes, hepatitis and the like) or parasites), in genetic or biochemical pathologies (e.g., cystic fibrosis, hemophilia, Alzheimer's disease, muscular dystrophy or multiple sclerosis) or in cancerous processes. Transformed or established animal cell lines may include, for example, COS cells, CHO cells, VERO cells, BHK cells, HeLa cells, HepG2 cells, K562 cells, 293 cells, L929 cells, F9 cells, and the like. Other cells, cell lines, tissues, organs and organisms suitable as sources of nucleic acids for use in the present invention will be apparent to one of ordinary skill in the art.

Once the starting cells, tissues, organs or other samples are obtained, nucleic acid molecules (such as rnRNA) may be isolated therefrom by methods that are well-known in the art (See, e.g., Maniatis, T., et al., Cell 15:687-701 (1978); Okayama, H., and Berg, P., Mol. Cell. Biol. 2:161-170 (1982); Gubler, U., and Hoffmnan, B. J., Gene 25:263-269 (1983); PCT Publication No. WO 98/08981; PCT Publication No. WO 98/51699; and PCT Publication No. WO 00/52191). The nucleic acid molecules thus isolated may then be used to prepare cDNA molecules and cDNA libraries in accordance with the present invention.

In the practice of the invention, cDNA molecules or cDNA libraries are produced by mixing one or more nucleic acid molecules obtained as described above, which is preferably one or more mRNA molecules such as a population of mRNA molecules, with one or more inhibitors of the present invention or with one or more of the compositions of the invention, under conditions favoring the reverse transcription of the nucleic acid molecule or molecules to form one or more cDNA molecules (single-stranded or double-stranded). Thus, the method of the invention comprises (a) mixing one or more nucleic acid templates (preferably one or more RNA or mRNA templates, such as a population of mRNA molecules) with one or inhibitors of the invention and more or more polypeptide having reverse transcriptase activity (b) incubating the mixture under conditions sufficient to make one or more nucleic acid molecules complementary to all or a portion of the one or more templates. Such methods may include the use of one or more DNA polymerases, one or more nucleotides, one or more primers, one or more buffers, and the like. The invention may be used in conjunction with methods of cDNA synthesis such as those described in the Examples below, or others that are well-known in the art (see, e.g., Gubler, U., and Hoffinan, B. J., Gene 25:263-269 (1983); Krug, M. S., and Berger, S. L., Meth. Enzymol. 152:316-325 (1987); Sambrook, J., et al., Molecular Cloning: A Laboratory Manual, 2nd ed., Cold Spring Harbor, N.Y.: Cold Spring Harbor Laboratory Press, pp. 8.60-8.63 (1989); PCT Publication No. WO 99/15702; PCT Publication No. WO 98/47912; PCT Publication No. WO 98/08981; PCT Publication No. WO 98/51699; and PCT Publication No. WO 00/52191; and PCT Publication No. WO 98/51699), to produce cDNA molecules or libraries.

Other methods of cDNA synthesis which may advantageously use the present invention will be readily apparent to one of ordinary skill in the art.

Having obtained cDNA molecules or libraries according to the present methods, these cDNAs may be isolated for further analysis or manipulation. Detailed methodologies for purification of cDNAs are taught in the GENETRAPPER™ manual (Invitrogen Corporation (Carlsbad, Calif.)), which is incorporated herein by reference in its entirety, although alternative standard techniques of cDNA isolation that are known in the art (see, e.g., Sambrook, J., et al., Molecular Cloning: A Laboratory Manual, 2nd ed., Cold Spring Harbor, N.Y.: Cold Spring Harbor Laboratory Press, pp. 8.60-8.63 (1989); U.S. Pat. No. 5,759,778; and U.S. patent appl. No. 2001/0036651) may also be used.

In other aspects of the invention, the invention may be used in methods for amplifying and sequencing nucleic acid molecules. Nucleic acid amplification methods according to this aspect of the invention may be one- step (e.g., one-step RT-PCR) or two-step (e.g., two-step RT-PCR) reactions. According to the invention, one-step RT-PCR type reactions may be accomplished in one tube thereby lowering the possibility of contamination. Such one-step reactions comprise (a) mixing a one or more nucleic acid template (e.g., mRNA) with one or more inhibitors of the present invention, one or more polypeptides having reverse transcriptase activity, and with one or more DNA polymerases and (b) incubating the mixture under conditions sufficient to amplify a nucleic acid molecule complementary to all or a portion of the templates. Such amplification may be accomplished by the reverse transcriptase activity alone or in combination with the DNA polymerase activity. Two-step RT-PCR reactions may be accomplished in two separate steps. Such a method comprises (a) mixing one or more nucleic acid templates (e.g., mRNA) with one or more inhibitors of the present invention and one or more polypeptides having reverse transcriptase activity (b) incubating the mixture under conditions sufficient to make a nucleic acid molecule (e.g., a DNA molecule) complementary to all or a portion of the templates, (c) mixing the nucleic acid molecule with one or more DNA polymerases and (d) incubating the mixture of step (c) under conditions sufficient to amplify the nucleic acid molecule. For amplification of long nucleic acid molecules (i.e., greater than about 3-5 Kb in length), a combination of DNA polymerases may be used, such as one DNA polymerase having 3′ exonuclease activity and another DNA polymerase being substantially reduced in 3′ exonuclease activity.

Nucleic acid sequencing methods according to this aspect of the invention may comprise both cycle sequencing (sequencing in combination with amplification) and standard sequencing reactions. The sequencing method of the invention thus comprises (a) mixing a nucleic acid molecule to be sequenced with one or more primers, one or more reverse transcriptases, one or more nucleotides and one or more terminating agents, (b) incubating the mixture under conditions sufficient to synthesize a population of nucleic acid molecules complementary to all or a portion of the molecule to be sequenced, and (c) separating the population to determine the nucleotide sequence of all or a portion of the molecule to be sequenced. According to the invention, one or more DNA polymerases (preferably thermostable DNA polymerases) may be used in combination with or separate from the reverse transcriptases. In one aspect, the nucleic acid molecules to be sequenced may be any of the nucleic acid molecules produced by the methods of the invention including cDNA molecules.

Amplification methods which may be used in accordance with the present invention include PCR (U.S. Pat. Nos. 4,683,195 and 4,683,202), Strand Displacement Amplification (SDA; U.S. Pat. No. 5,455,166; EP 0 684 315), and Nucleic Acid Sequence-Based Amplification (NASBA; U.S. Pat. No. 5,409,818; EP 0 329 822), as well as more complex PCR-based nucleic acid fingerprinting techniques such as Random Amplified Polymorphic DNA (RAPD) analysis (Williams, J. G. K., et al., Nucl. Acids Res. 18(22):6531-6535, 1990), Arbitrarily Primed PCR (AP-PCR; Welsh, J., and McClelland, M., Nucl. Acids Res. 18(24):7213-7218, 1990), DNA Amplification Fingerprinting (DAF; Caetano-Anollés et al., Bio/Technology 9:553-557, 1991), microsatellite PCR or Directed Amplification of Minisatellite-region DNA (DAMD; Heath, D. D., et al., Nucl. Acids Res. 21(24): 5782-5785, 1993), and Amplification Fragment Length Polymorphism (AFLP) analysis (EP 0 534 858; Vos, P., et al., Nucl. Acids Res. 23(21):4407-4414, 1995; Lin, J. J., and Kuo, J., FOCUS 17(2):66-70, 1995). Nucleic acid sequencing techniques which may employ the present compositions include dideoxy sequencing methods such as those disclosed in U.S. Pat. Nos. 4,962,022 and 5,498,523. In a particularly preferred aspects, the invention may be used in methods of amplifying or sequencing a nucleic acid molecule comprising one or more polymerase chain reactions (PCRs), such as any of the PCR-based methods described above.

### Formulation of Composition or Reaction Mixtures

To form the compositions or reaction mixtures of the present invention, one or more inhibitors are preferably admixed in a buffered salt solution. The compositions of the invention may also comprise one or more metal ions or metal salts such as Mg⁺⁺, Mn⁺⁺, MgCL₂, MnCl₂, K⁺, or KCL. One or more reverse transcriptases, one or more DNA polymerases and/or one or more nucleotides, and/or one or more primers may optionally be added to make the compositions of the invention. The water used in forming the compositions of the present invention is preferably distilled, deionized and sterile filtered (through a 0.1-0.2 micrometer filter), and is free of contamination by DNase and RNase enzymes. Such water is available commercially, for example from Sigma Chemical Company (Saint Louis, Mo.), or may be made as needed according to methods well known to those skilled in the art.

In addition to the inhibitor components, the present compositions preferably comprise one or more buffers and cofactors necessary for synthesis of a nucleic acid molecule such as a cDNA molecule. Particularly preferred buffers for use in forming the present compositions are the acetate, sulfate, hydrochloride, phosphate or free acid forms of Tris-(hydroxymethyl)aminomethane (TRIS(®), although alternative buffers of the same approximate ionic strength and pKa as TRIS® may be used with equivalent results.

### Inhibitor/Degradation Component Ratio

According to a preferred aspect of the present invention, the ratio of inhibitor to degradation component in the reaction mixtures, compositions and methods of the invention may be optimized to minimize the amount of RNA degradation during nucleic acid synthesis. Preferred molar ratios of inhibitor to degradation component range from about 1000:1; 750:1; 500:1; 250:1; 100:1; 75:1; 50:1; 25:1; 12:1; 11:1; 10:1; 9:1; 8:1; 7:1; 6:1; 5:1; 4:1; 3:1; 2:1; 1:1; 1:2; 1:3; 1:4; 1:5; 1:6; 1:7; 1:8; 1:9; 1:10; 11:1; 1:12; 1:25; 1:50; 1:75; 1:100; 1:250; 1:500; 1:750; and 1:1000. Preferably, molar ratios of inhibitor to degradation component range from about 2:1 to about 1:20, although lower or higher molar ratios of inhibitor to degradation component may be used in accordance with the invention. Specifically, molar ratios of inhibitor to degradation component may be below about 1:10; 1:15; 1:20; 1:25; 1:50; 1:75; and 1:100. Preferably, ranges of molar ratios are below 5:1; 4:1, 3:1; 2:1; 1:1; 1:2; 1:3; 1:4; and 1:5. Most preferably, ratios of inhibitor to degradation component range from about 1:1 to 2:1; 1:1.5 to 1:1; 1:2 to 0.5:1; 1:2.5 to 0.25:1; 1:3 to 0.10:1; 1:2 to 1:1; 1:1 to 1.5:1; 1:0.5 to 2:1; 1:0.25 to 2.5:1; and 1:0.10 to 3:1.0; 10:1 to 1:10; 5:1 to 1:10; 4:1 to 1:10; 3:1 to 1:10; 2.5:1 to 1:10; 2:1 to 1:10; 1.5:1 to 1:10; and 1:1 to 1:10. The optimum ratios of inhibitor to degradation component may vary depending on the inhibitor, the template, the primer, the reverse transcription enzyme and/or other reaction conditions used. The desired ratio of inhibitor to degradation component can be readily determined by one skilled in the art.

### Inhibitor/Nucleic Acid Template Ratio

The inhibitor of the present invention are preferably used in the present reaction mixtures, compositions and methods at various inhibitor/template ratios. Such ratios may be optimized by one skilled in the art to minimize the amount of RNA degradation in a nucleic acid synthesis reaction. The ratio of the number of inhibitors or amounts of inhibitors to the number of templates or amount of templates may vary depending on the RT used. The ratio of inhibitor to template may range from about 0.001-100:1; 0.01 1000:1; 0.1-10,000:1; 1-100,000:1; 1:0.001-100; 1:0.01-1000; 1:0.1-10,000; or 1:1-100,000. Of course, other suitable ratios of such inhibitor to template suitable for use in the invention will be apparent to one of ordinary skill in the art or determined with no more than routine experimentation.

### Concentrations of Inhibitors

In a preferred aspect of the invention, the concentration of the inhibitors will be at least 10 micromolar, at least 25 micromolar, at least 50 micromolar, at least 75 micromolar, at least 100 micromolar, at least 200 micromolar, at least 300 micromolar, at least 400 micromolar, at least 500 micromolar, at least 600 micromolar, at least 700 micromolar, at least 800 micromolar, at least 900 micromolar, at least 1000 micromolar, at least 1500 micromolar, at least 2000 micromolar, at least 2100 micromolar, at least 2200 micromolar, at least 2300 micromolar, at least 2400 micromolar, at least 2500 micromolar, at least 2600 micromolar, at least 2700 micromolar, at least 2900 micromolar, at least 3000 micromolar, at least 3500 micromolar, at least 4000 micromolar, at least 4500 micromolar, at least 5000 micromolar, at least 5500 micromolar, at least 6000 micromolar, at least 6500 micromolar, at least 7000 micromolar, at least 7500 micromolar, at least 8000 micromolar, at least 8500 micromolar, at least 9000 micromolar, at least 9500 micromolar, at least 10,000 micromolar, at least 10,500 micromolar, at least 11,000 micromolar, at least 11,500 micromolar, at least 12,000 micromolar, at least 12,500 micromolar, at least 13,000 micromolar, at least 13,500 micromolar, at least 14,000 micromolar, at least 14,500 micromolar, at least 15,000 micromolar, at least 15,500 micromolar, at least 16,000 micromolar, at least 16,500 micromolar, at least 17,000 micromolar, at least 17,500 micromolar, at least 18,000 micromolar, at least 18,500 micromolar, at least 19,000 micromolar, at least 19,500 micromolar, or at least 20,000 micromolar.

In a preferred aspect of the invention, the range of concentration of the inhibitors will be at least 10-20,000 micromolar, at least 25-19,500 micromolar, at least 50-19,000 micromnolar, at least 75-18,500 micromolar, at least 100-18,000 micromolar, at least 200-17,500 micromolar, at least 300-17,000 micromolar, at least 400-16,500 micromolar, at least 500-16,000 micromolar, at least 600-15,500 micromolar, at least 700-15,000 micromolar, at least 800-14,500 micromolar, at least 900-14,000 micromolar, at least 1000-13,500 micromolar, at least 1500-13,500 micromolar, at least 1750-13,000 microrrolar, at least 2000-12,500 micromolar, at least 2000-12,000 micromolar, at least 2100.-11,500 micromolar, at least 2200-11,000 micromolar, at least 2300- 10,500 micromolar, at least 2400-10,000 micromolar, at least 2500- 9,500 micromolar, at least 2600-9,000 micromolar, at least 2700-8,500 micromolar, at least 2800-8,000 micromolar, at least 2900-7,500 micromolar, at least 3000-7,000 micromolar, at least 3500-6,500 micromolar, at least 4000-6,000 micrormolar, at least 4500-5,500 micromolar, at least 5000-5,000 micromolar, at least 5500-4,500 micromolar, at least 6,000-4,000 micromolar, at least 6,500-3,500 micromolar, at least 7,000-3,000 micromolar, at least 8,000-2,900 micromolar, at least 8,500-2,800 micromolar, at least 9,000-2,700 micromolar, at least 9,500-2,600 micromolar, at least 10,000-2,500 micromolar, at least 10,500-72,400 micromolar, at least 11,000-2,300 micromolar, at least 11,500-2,200 micromolar, at least 12,000-2,100 micromolar, at least 12,500-2,000 micromolar, at least 13,000- 2,000 micromolar, at least 13,500- 1,500 micromolar, at least 14,000-1,000 micromolar, at least 14,500-900 micromolar, at least 15,000-800 micromolar, at least 15,500-700 micromolar, at least 16,000-600 micromolar, at least 16,500- 500 micromolar, at least 17,000-400 micromolar, at least 17,500-300 micromolar, at least 18,000-200 micromolar, at least 18,500-100 micromolar, at least 19,000-75 micromolar, at least 19,500-50 micromolar, at least 20,000-25 micromolar, or at least 20,000-10 micromolar.

In a preferred aspect of the invention, the concentration of the inhibitors will be at least 2,100-20,000 micromolar, at least 2,200-20,000 micromolar, at least 2,300-20,000 micromolar, at least 2,400-20,000 micromolar, at least 2,500-20,000 micromolar, at least 2,600-20,000 micromolar, at least 2,700-20,000 micromolar, at least 2,800-20,000 micromolar, at least 2,900-20,000 micromolar, at least 3,000-20,000 micromolar, at least 3,100-20,000 micromolar, at least 3,200-20,000 micromolar, at least 3,300-20,000 micromolar, at least 3,400-20,000 micromolar, at least 3,500-20,000 micromolar, at least 3,600-20,000 micromolar, at least 3,700-20,000, at least 3,800-20,000 micromolar, at least 3,900-20,000 micromolar, and most preferably a concentration of at least 4,000 to 20,000 micromolar.

### Kits

In another embodiment, the present invention may be assembled into kits for use in reverse transcription or amplification of a nucleic acid molecule, or into kits for use in sequencing of a nucleic acid molecule. Kits according to this aspect of the invention comprise a carrier means, such as a box, carton, tube or the like, having in close confinement therein one or more container means, such as vials, tubes, ampoules, bottles and the like, wherein a first container means contains one or more inhibitors. When more than one inhibitor is used, they may be in a single container as mixtures of two or more inhibitors, or in separate containers. The kits of the invention may also comprise one or more polypeptides having reverse transcriptase activity. When more than one polypeptide having reverse transcriptase activity is used, they may be in a single container as mixtures of two or more polypeptides, or in separate containers. The kits of the invention may also comprise (in the same or separate containers) one or more DNA polymerases, a suitable buffer, one or more nucleotides and/or one or more primers.

In a specific aspect of the invention, the reverse transcription and amplification kits may comprise one or more components (in mixtures or separately) including one or more inhibitors of the invention, one or more polypeptides having reverse transcriptase activity, one or more nucleotides needed for synthesis of a nucleic acid molecule, and/or one or more primers (e.g., oligo(dT) for reverse transcription). Such reverse transcription and amplification kits may further comprise one or more DNA polymerases. Sequencing kits of the invention may comprise one or more polypeptides having reverse transcriptase activity, and optionally one or more DNA polymerases, one or more terminating agents (e.g., dideoxynucleoside triphosphate molecules) needed for sequencing of a nucleic acid molecule, one or more nucleotides and/or one or more primers. Preferred inhibitors of the invention, polypeptides having reverse transcriptase activity, DNA polymetases, nucleotides, primers and other components suitable for use in the reverse transcription, amplification and sequencing kits of the invention include those described above. The kits encompassed by this aspect of the present invention may further comprise additional reagents and compounds necessary for carrying out standard nucleic acid reverse transcription, amplification or sequencing protocols. Such inhibitors of the invention, polypeptides having reverse transcriptase activity, DNA polymerases, nucleotides, primers, and additional reagents, may be contained in one or more containers, and may be contained in such containers in a mixture of two or more of the above-noted components or may be contained in the kits of the invention in separate containers. The kits of the invention may also comprise one or more host cells, preferably cells which are capable of uptake of nucleic acid molecules or vectors (such as chemically competent host cells and electrocompetent host cells). Preferred host cells for use in the kits of the invention include chemically competent E.coli cells (such as UltraMAX™ DH5α-FT™, MAX Efficiency® DH5α™. T1 Phage-Resistant, and One Shot® MAX Efficiency® DH10B™ T1 Phage Resistant) and electrocompetent E.coli cells (such as ElectroMAX™ DH10B™ T1 Phage Resistant, ElectroMAX™ DH10B™, Top10 Electrocomp™, One Shot® TOP 10 Electrocomp™, and One Shot® GeneHogs® Electrocomp™), both of which are available from Invitrogen Corporation.

### Use of Nucleic Acid Molecules

The nucleic acid molecules or cDNA libraries prepared by the methods of the present invention may be further characterized, for example by cloning and sequencing (i.e., determining the nucleotide sequence of the nucleic acid molecule), by the sequencing methods of the invention or by others that are standard in the art (see, e.g., U.S. Pat. Nos. 4,962,022 and 5,498,523, which are directed to methods of DNA sequencing). Alternatively, these nucleic acid molecules may be used for the manufacture of various materials in industrial processes, such as hybridization probes by methods that are well-known in the art. Production of hybridization probes from cDNAs will, for example, provide the ability for those in the medical field to examine a patient's cells or tissues for the presence of a particular genetic marker such as a marker of cancer, of an infectious or genetic disease, or a marker of embryonic development. Furthermore, such hybridization probes can be used to isolate DNA fragments from genomic DNA or cDNA libraries prepared from a different cell, tissue or organism for further characterization.

The nucleic acid molecules of the present invention may also be used to prepare compositions for use in recombinant DNA methodologies. Accordingly, the present invention relates to recombinant vectors which comprise the cDNA or amplified nucleic acid molecules of the present invention, to host cells which are genetically engineered with the recombinant vectors, to methods for the production of a recombinant polypeptide using these vectors and host cells, and to recombinant polypeptides produced using these methods.

Recombinant vectors may be produced according to this aspect of the invention by inserting, using methods that are well-known in the art, one or more of the cDNA molecules or amplified nucleic acid molecules prepared according to the present methods into a vector. The vector used in this aspect of the invention may be, for example, a phage or a plasmid, and is preferably a plasmid. Preferred are vectors comprising cis-acting control regions to the nucleic acid encoding the polypeptide of interest. Appropriate trans-acting factors may be supplied by the host, supplied by a complementing vector or supplied by the vector itself upon introduction into the host.

In certain preferred embodiments in this regard, the vectors provide for specific expression (and are therefore termed “expression vectors”), which may be inducible and/or cell type-specific. Particularly preferred among such vectors are those inducible by environmental factors that are easy to manipulate, such as temperature and nutrient additives.

Expression vectors useful in the present invention include chromosomal-, episomal- and virus-derived vectors, e.g., vectors derived from bacterial plasmids or bacteriophages, and vectors derived from combinations thereof, such as cosmids and phagemids, and will preferably include at least one selectable marker such as a tetracycline or ampicillin resistance gene for culturing in a bacterial host cell. Prior to insertion into such an expression vector, the cDNA or amplified nucleic acid molecules of the invention should be operatively linked to an appropriate promoter, such as the phage lambda P_{L} promoter, the E. coli lac, trp and tac promoters. Other suitable promoters will be known to the skilled artisan.

Among vectors preferred for use in the present invention include pQE70, pQE60 and pQE-9, available from Qiagen; pBS vectors, Phagescript vectors, Bluescript vectors, pNH8A, pNH16a, pNH18A, pNH46A, available from Stratagene; pcDNA3 available from Invitrogen; pGEX, pTrxfus, pTrc99a, pET-5, pET-9, pKK223-3, pKK233-3, pDR540, pRIT5 available from Pharmacia; and Gateway™ vectors such as pET-DEST42, pDESTTM™14, pBAD-DEST49, pYES-DEST52, pDEST™8, pcDNA-DEST40, pT-REx-DEST30, pENTR 1A, pENTR 2B, pENTR3C, pDONR™ 201, pDONR™ 207, and other vectors such as pSPORT1, pSPORT2 and pSV.SPORT1, pSPORT6, available from Invitrogen Corporation (Carlsbad, Calif.). Other suitable vectors will be readily apparent to the skilled artisan.

The invention also provides methods of producing a recombinant host cell comprising the cDNA molecules, amplified nucleic acid molecules or recombinant vectors of the invention, as well as host cells produced by such methods. Representative host cells (prokaryotic or eukaryotic) that may be produced according to the invention include, but are not limited to, bacterial cells, yeast cells, plant cells and animal cells. Preferred bacterial host cells include Escherichia coli cells (most particularly E. coli strains DH10B and Stb12, which are available commercially (Invitrogen Corporation (Carlsbad, Calif.)), Bacillus subtilis cells, Bacillus megaterium cells, Streptomyces spp. cells, Erwinia spp. cells, Klebsiella spp. cells and Salmonella typhimurium cells. Preferred animal host cells include insect cells (most particularly Spodoptera frugiperda Sf9 and Sf21 cells and Trichoplusa HigH-Five cells) and mammalian cells (most particularly CHO, COS, VERO, BHK and human cells). Such host cells may be prepared by well-known transformation, electroporation or transfection techniques that will be familiar to one of ordinary skill in the art. Preferred host cells are cells made competent for uptake of nucleic acid molecules such as chemically competent cells and electrocompetent cells.

In addition, the invention provides methods for producing a recombinant polypeptide, and polypeptides produced by these methods. According to this aspect of the invention, a recombinant polypeptide may be produced by culturing any of the above recombinant host cells under conditions favoring production of a polypeptide therefrom, and isolation of the polypeptide. Methods for culturing recombinant host cells, and for production and isolation of polypeptides therefrom, are well-known to one of ordinary skill in the art.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

Reverse Transcription with M-MLV H⁻ RT and RSV H⁻ RT at Elevated Temperatures in Combination with an Inhibitor

Historically, it has been shown that the efficiency and specificity of the cDNA synthesis from mRNA catalyzed by reverse transcriptase (RT) are improved by increasing the temperature above 40° C. However, increasing the temperature of the reaction commonly leads to accelerated Mg2+-catalyzed RNA hydrolysis during cDNA synthesis. To alleviate this problem, it is desirable to add one or more inhibitors of the invention to the synthesis reaction. This example describes the use of excess dNTP's to reduce RNA degradation during cDNA synthesis.

General Methods

Enzymes

SuperScript™ II RT, a cloned form of Moloney murine leukemia virus (M-MLV) RT lacking demonstrable RNase H activity (i.e., an RNase H⁻ RT), was obtained from Invitrogen Corporation (Carlsbad, Calif.). ThermoScript™, a genetically engineered avian reverse transcriptase with greatly reduced RNase H activity and high thermal stability, was also from Invitrogen. A mutant M-MLV H⁻ RT with increased thermal stability was generated and purified as described in U.S. Pat. No. 5,017,492, U.S. appl. Ser. No. 09/845,157, filed May 1, 2001, U.S. appl. Ser. Nos. 60/207,196 and 60/410,283, PCT Publication No. WO 01/92500, and PCT Appl. No. US01/16861 (the entire disclosure of which is incorporated herein by reference).

RNA

RNA (7.5Kb) was labeled at the 3′ end with [α-³²P]ddATP (Amersham Pharmacia) and yeast poly(A) polymerase (Amersham Pharmacia) following the manufacturer's protocol. MAP-4 cRNA (5.2 kb) was synthesized by T7 RNA polymerase run-off transcription from linearized plasmid DNA (D'Alessio et al., Nuc. Acids Res. 16, 1999-2014, 1988). The sequence at the 3′ end of the cRNA if run-off transcription is complete is 5′-(N)ₙ-(A)₄₀-UAAAGUAUAUUACCA-3′. The cRNA was selected on oligo(dT)-cellulose to ensure the presence of a poly(A) tail.

RNA Heating Procedure

RNA was heated at various temperatures in a 0.5-ml tube in a thermocycler equipped with a heated lid. The heating solution (20 μl), designed to mimick M-MLV RT reaction conditions, contained 50 mM Tris-HCl (pH 8.4), 75 mM KCl, 3 mM MgCl₂, 500 μM each of dCTP, dGTP, dTTP, and dATP, and 10 μg of unlabeled or 3′-end labeled 7.5-Kb RNA. At various times, 2-μl aliquots were removed and placed on ice in separate tubes containing 1 μl of 100 mM EDTA. Gel loading buffer (7 μl) was added and the RNA was heated at 65° C. for 5 min. When testing avian RT reaction conditions, the MgCl₂ concentration was increased to 7.5 mM and the concentration of each dNTP was increased to 1 mM in heating -mixtures.

Gel Electrophoresis

Heated RNA was subjected to denaturing formaldehyde agarose gel electrophoresis as described (Gerard et al. Focus 8, 5-6, 1987). Gels contained 1.5% agarose, 50 mM MOPS (pH 7.0), 1 mM EDTA, and 2.2 M formaldehyde and were run in 50 mM MOPS (pH 7.0)-1 mM EDTA.

Gel Analysis

A digitized image of EtBr-stained unlabeled RNA was captured during transillumination of gels with short wave TV light with a Lynx 5000 Gel Imaging System (Applied Imaging, CA). The system software was used to quantify the fraction of intact 7.5-Kb RNA present in a lane of the gel. When labeled RNA was used, the gel was dried and exposed to x-ray film. Using an exposed film as a guide, the intact 7.5-kb RNA and any breakdown products were excised separately and counted in a scintillation counter to determine the fraction of intact RNA.

cDNA Synthesis Reaction Mixtures

Reaction mixtures (20 μl each) contained the following components unless specified otherwise: 50 mM Tris-HCl (pH 8.4), 75 mM KCl, 3 mM MgCl₂, 10 mM dithiothreital (DTT), 500 μM each of dATP, dTTP, dGTP, and [α-³²P]dCTP (400 cpm/pmole), 1,750 units/ml RNase Inhibitor, 50 μg/ml (62nM) 5.2-Kb MAP-4 cRNA, 5 μg/ml (575 nM) p(dT)₂₅₋₃₀. These mixtures were combined with the following amounts and types of RT:

| | |
|---|---|
| M-MLV H⁻ RT | 10,000 units/ml (430 nM) |
| RSV H⁻ RT | 750 units/ml (80 nM) |

In the reaction mixtures for RSV H⁻ RT the MgCl₂ and individual dNTP concentrations were increased to 7.5mM and 1mM, respectively. In some cases, the concentration of each dNTP was increased to 1mM or 2mM.

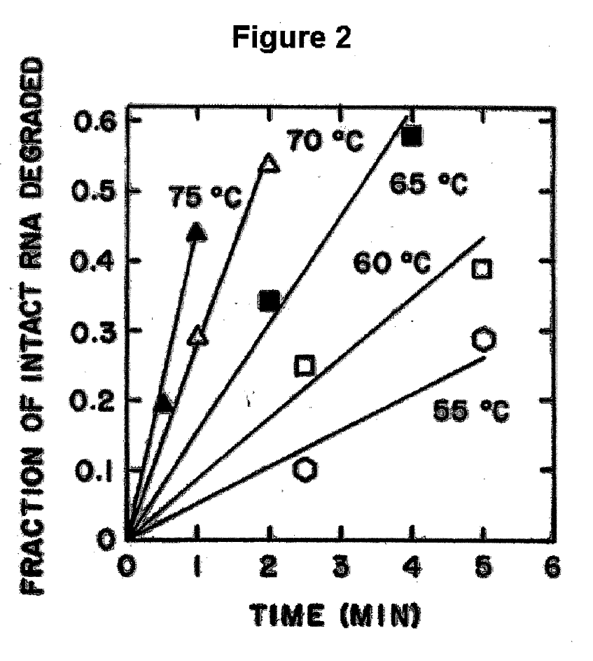

The cDNA synthesis reactions were carried out at either 45° C. or 55° C. for 60 minutes. Incubation was at the temperatures and for the times indicated in FIG. 2. An aliquot of the reaction mixture was precipitated with TCA to determine total yield of cDNA synthesized, and the remaining cDNA product was size fractionated on an alkaline 1.2% agarose gel (McDonell et al., J. Mol. Biol. 110, 119-146, 1977). The yield of full-length cDNA was determined by using x-ray film exposed to a dried gel as a guide to cut and count gel sections in a scintillation counter.

Rate of Loss and Half-Life Determinations

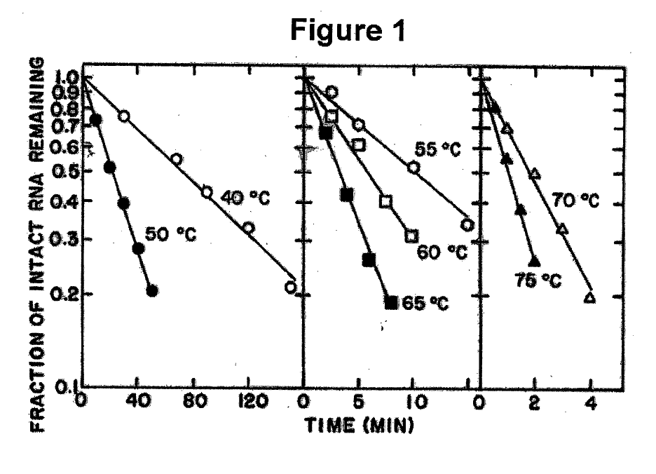

The breakdown of RNA was monitored by denaturing, agarose gel electrophoresis. The conditions selected were those optimal for M-MLV RT (Gerard et al., Molecular Biotechnology 8, 61-77, 1997) and contained 1mM Mg2⁺ in excess over a total dNTP concentration of 2mM. At various temperatures, an initial rate of loss of intact 7.5-Kb RNA was determined, as well as RNA half-lives over extended periods. Either unlabeled or ³²P-labeled 7.5-Kb cRNA was used. The results obtained with labeled and unlabeled RNA were similar. The loss of intact 7.5-Kb RNA at various temperatures as a function of time in the presence of 1 mM excess Mg²⁺ is shown in FIG. 1. The fraction of 7.5-Kb RNA remaining intact at various temperatures was plotted on a logarithmic scale versus time in minutes on a linear scale at temperatures ranging from 40 to 75° C.

TABLE 1
Half-lives and Rates of Loss of Intact 7.5-Kb cRNA Exposed to
M-MLV RT Reaction Conditions at Various Temperatures^{a}

| Temperature | Half-life^{b} | Rate of Loss of Intact cRNA^{c} |
|---|---|---|
| (° C.) | (min) | (fraction/min) |
| 40 | 67 (62, 72) | 0.01 (0.01, 0.01) |
| 50 | 21 (21, 21) | 0.03 (0.03, 0.03) |
| 55 | 10 (10, 10) [5.0]^{d} | 0.05 (0.05, 0.05) [0.11]^{d} |
| 60 | 6.1 (6.0, 6.2) | 0.10 (0.09, 0.11) |
| 65 | 2.9 (2.8, 3.0) | 0.16 (0.16, 0.17) |
| 70 | 1.8 (1.8, 1.8) | 0.27 (0.27, 0.27) |
| 75 | 0.8 (0.7, 0.9) | 0.49 (0.42, 0.56) |

^{a}Mixtures contained 3 mM MgCl₂ and 2 mM total dNTPs
^{b}Mean of two determinations is shown with individual values in parentheses.
^{c}Mean of two determinations is shown with individual values in parentheses. The rate of loss of intact cRNA is the fraction of intact RNA lost per min calculated from the slope of the straight line generated from the first two data points at each temperature in FIG. 1 plotted in FIG. 2 as fraction of intact RNA lost versus time.
^{d}The values in brackets were determined in the presence of 7.5 mM MgCl₂ and 4 mM total dNTPs.

The first two data points from each time course were also used to calculate a rate of loss of intact 7.5-Kb RNA that is expressed as the fraction of intact RNA lost per min at each temperature (FIG. 2 and Table 1). The initial rates of hydrolysis of intact RNA expressed as fraction lost/min ranged from 0.01/min at 40° C. to 0.49/min at 75° C. On average, the rate of RNA breakdown increased 1.75-fold for every 5° C. increment in temperature increase above 40° C. in the presence of 1 mM excess Mg²⁺.

Optimal reaction conditions for avian RT contain 7.5 mM MgCl₂ and 4 mM total dNTPs or 3.5 mM Mg²⁺ in excess over total dNTPs. (Gerard et al., Nuc. Acids Res., 2002; Schwabe et al., Focus 20, 30-33, 1999) The half-life and rate of breakdown of 7.5 kb RNA using avain RT were determined at 55° C. under these conditions.

Relative to M-MLV RT reaction conditions, the half-life was reduced from 10 to 5 min and the rate of breakdown more than doubled from 0.05 to 0.11 fraction degraded/min when avain RT is used (Table 1). The faster breakdown rate reflects the higher concentration of free Mg²⁺ (Barshevskaya et al., Molekulyamaya Biologiya 21, 1235-1241, 1987).

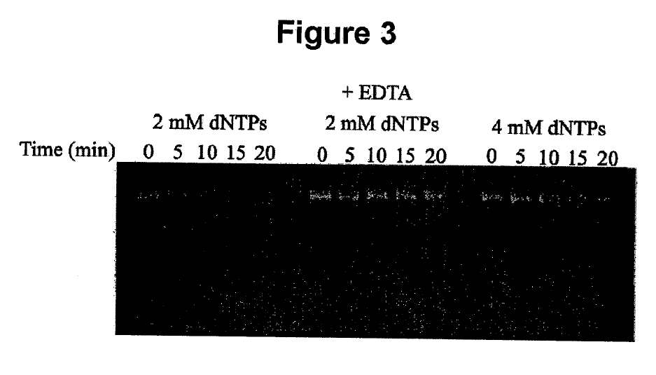

Since the RNA breakdown observed requires free Mg²⁺ and is proportional to free Mg²⁺ concentration, it might be possible to inhibit the reaction by reducing or eliminating free Mg²⁺. FIG. 3 shows that this can be accomplished at 60° C. by addition of excess EDTA. We hypothesized that it should also be possible by addition of dNTPs in excess over MgCl₂ to chelate free Mg²⁺ ion and thus protect RNA from breakdown. FIG. 3 also shows that when 4 mM dNTPs were present with 3 mM MgCl₂, there was very little breakdown of 7.5-Kb RNA at 60° C. during a 20-min incubation.

Effect of High dNTP Concentration on cDNA Synthesis at Elevated Temperatures

If RT does not require free Mg²⁺ to catalyze efficient cDNA synthesis, it should be possible to protect mRNA from Mg²⁺-catalyzed degradation without compromising the efficiency of cDNA synthesis. Table 2 shows the results at 45° C. of altering the M-MLV H−RT and RSV H−RT optimal reaction conditions to include a total dNTP concentration that exceeds the MgCl₂ concentration.

TABLE 2
The Effect of High dNTP Concentration on cDNA Synthesis
at 45° C.

| | | Yield of cDNA Synthesized^{a,b} | |
|---|---|---|---|
| Enzyme | Total [dNTP] (mM) | Total (ng) | Full Length (ng) |
| M-MLV H-RT | 2 | 262 ± 15 | 124 ± 8 |
| M-MLV H-RT | 4 | 283 ± 2 | 126 ± 6 |
| RSV H-RT | 4 | 308 ± 8 | 106 ± 5 |
| RSV H-RT | 8 | 255 ± 15 | 86 ± 10 |

^{a}cDNA was synthesized from MAP-4 5.2-Kb cRNA at 45° C. for 60 min and the product was analyze as described in Materials and Methods. M-MLV H-RT (1 μl, 200 units) or RSV H-RT (1 μl, 15 units) was added to initiate the reaction.
^{b}The mean ± standard deviation is shown of four to five individual determinations.

The rate of Mg²⁺-catalyzed RNA breakdown at this temperature is minimal so that the results were not appreciably influenced by RNA hydrolysis. The total yield of cDNA synthesized from 5.2-Kb cRNA by M-MLV H−RT increased slightly while the total yield synthesized by RSV H−RT decreased at the higher dNTP concentration. There was no change in the yield of full-length product synthesized by M-MLV H- RT. As with the total yield, the yield of full-length product synthesized by RSV H- RT was reduced slightly at the higher dNTP concentration. This reduction was observed reproducibly in other experiments. We conclude that the efficiency of cDNA synthesis by M-MLV H−RT is not reduced by increasing the total dNTP concentration from 2 to 4 mM. The potential benefit of chelating free Mg2⁺ with excess dNTPs in a RT reaction at a moderately elevated temperature is indicated by the results in Table 3.

TABLE 3
The Effect of High dNTP Concentration on cDNA Synthesis
at 55° C.

Yield of cDNA Synthesized^{a,b}

| Total [dNTP] (mM) | Total (ng) | Full Length (ng) |
|---|---|---|
| 2 | 192 ± 11 | 27 ± 3 |
| 4 | 186 ± 16 | 38 ± 12 |

^{a}cDNA was synthesized from MAP-4 5.2-Kb cRNA at 55° C. and the product was analyzed as described in Materials and Methods. A thermal stable mutant of M-MLV H-RT (1 μl, 200 units) was added to the reaction at 15-min intervals during a 60-min incubation.

A thermal stable mutant of M-MLV H−RT was used to synthesize ³²P-labeled cDNA from 5.2 kb cRNA at 55° C. Synthesis was carried out either under normal reaction conditions with 1 mM excess Mg²⁺ or under modified conditions with total dNTPs in excess over Mg²⁺ by 1 mM. Total yield of cDNA synthesized was comparable. A modest increase in full-length 5.2 kb cDNA synthesized at 55° C. was observed when the total dNTP concentration exceeded that of Mg²⁺.

Mg²⁺-catalyzed RNA degradation occurs at a relatively rapid rate in the presence of just 1 mM excess Mg²⁺ at temperatures above 50° C. At 55° C., for example, half the RNA is no longer intact after 10 min. Depending upon the sequence of the RNA copied, the chain elongation rate of retroviral RT varies between 5 and 30 nucleotides/sec at 37 to 45° C. (Gerard et al., Nuc. Acids Res., 2002; Kelleher et al., J. Biol. Chem. 273, 9976-9986, 1998; Majumdar et al., J. Biol. Chem. 263, 15657-15665, 1988). Representatives of most mRNAs would probably survive intact long enough under these RT reaction conditions and with these chain growth rates to be copied to their 5′ ends. However, RT tends to pause during CDNA synthesis at certain sequences in mRNA not involved in secondary structure (Klarmann et al., J. Biol. Chem. 268, 9793-9802, 1993; Lavigne et al., J. Mol. Biol. 285, 977-995, 1999). The chain elongation rate drops to near zero at these sites (DeStefano et al., Biochim. Biophys. Acta 1131, 270-280, 1992). RNA possessing such sites might not be copied to full length at 55° C. before being cleaved. In addition, as the length of mRNA increases the number of such sites would tend to accumulate, decreasing even further the chances of achieving full-length synthesis before mRNA breakdown. On the other hand, at 55° C. some secondary structural elements would begin to melt, potentially increasing the overall rate of chain elongation by RT. At 65° C., half the RNA is no longer intact after 3 min. The chances of achieving full-length copying of most mRNA species are reduced. At this temperature applications such as RT-PCR that generally require the synthesis of shorter cDNA copies could be carried out in the presence of excess Mg²⁺, but cDNA library preparation would be problematic.

When excess Mg²⁺ is chelated with dNTP substrate, the stability of RNA increases and M-MLV H−RT can function efficiently with dNTPs present in excess over Mg²⁺. At 55° C. an increase in full-length synthesis of a long 5.2-Kb cRNA was obtained in the presence of excess dNTPs with a thermostable mutant of M-MLV H- RT that can operate at 55° C. This approach has potential for facilitating the synthesis of long cDNAs without metal ion catalyzed RNA breakdown at even higher temperatures with other mutant forms of retroviral RT that function at higher temperatures. Thermostable DNA polymerases from thermophiles have been reported to possess RT activity and can be used optimally at 60 to 70° C. (Myers et al., Biochem. 30, 7661-7666, 1991; Myers et al., Amplification of RNA: High-temperature reverse transcription and DNA amplification with Thermus thermophilus DNA polymerase. p. 58-68, In M. A. Innis, D. H. Gelfand, and J. J. Sninsky (Eds.), PCR Strategies, Academic Press, N.Y., 1995.) Those thermophile-derived DNA polymerases possessing RT activity generally fall into the E. coli DNA polymerase I-like family (Abramson, R. D., Thermostable DNA polymerases. p. 39-57. In M. A. Innis, D. H. Gelfand, and J. J. Sninsky (Eds.), PCR Strategies, Academic Press, N.Y., 1995).

Several of these DNA polymerases were tested for the ability to copy RNA with Mg2⁺ or dNTPs in excess. In contrast to M-MLV H minus RT, these DNA polymerases appear to require free Mg²⁺ for optimal activity and thus probably cannot be optimally used at 60-70° C. under conditions of excess dNTPs that would protect an RNA template from Mg²⁺-catalyzed hydrolysis. In any event, other inhibitors of the invention can be efficiently used with these DNA polymerases in accordance with the invention.

Having now fully described the present invention in some detail by way of illustration and example purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents, and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. An inhibitor to prevent, reduce, substantially reduce, or eliminate degradation of nucleic acid templates during nucleic acid synthesis.

2. The inhibitor of claim 1, wherein said inhibitor has a net negative charge.

3. The inhibitor of claim 1, wherein said inhibitor is not a ribonuclease inhibitor.

4. The inhibitor of claim 2, wherein said inhibitor is not a protease.

5. The inhibitor of claim 2, wherein said inhibitor is a chemical compound.

6. The inhibitor of claim 3, wherein said inhibitor is one or more nucleotides.

7. The inhibitor of claim 6, wherein said inhibitor interacts with or binds to a degradation component.

8. The inhibitor of claim 7, wherein said degradation component is Mg⁺⁺ or a salt thereof and said inhibitor is one or more nucleotides.

9. A composition for use in reverse transcription of a nucleic acid molecule, said composition comprising one or more inhibitors and one or more degradation components.

10. The composition of claim 8, further comprising one or more polypeptides having reverse transcriptase activity.

11. The composition of claim 8, wherein said polypeptides are reduced or substantially reduced or lacking in RNase H activity.

12. The composition of claim 8, wherein said polypeptides are selected from the group comprising M-MLV reverse transcriptase, ASV reverse transcriptase, HIV reverse transcriptase, Avian Sarcoma-Leukosis Virus (ASLV) reverse transcriptase, Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase, Avian Sarcoma Virus Y73 Helper Virus YAV reverse transcriptase, Rous Associated Virus (RAV) reverse transcriptase, and Myeloblastosis Associated Virus (MAV) reverse transcriptase and derivatives, variants, or fragments having reverse transcriptase activity or mutants thereof.

13. A method for reverse transcription of one or more nucleic acid molecules comprising:
(a) mixing one or more nucleic acid templates with one or more inhibitors and one or more polypeptides having reverse transcriptase activity; and
(b) incubating mixture of (a) under conditions sufficient to make one or more first nucleic acid molecules complementary to all or a portion of said one or more templates.

14. The method of claim 14, wherein said nucleic acid template is a messenger RNA molecule, a poly A⁺ RNA molecule, or a population of mRNA molecules.

15. The method of claim 14, wherein said mixture is incubated at temperatures ranging from 40° C. to 75° C.

16. The method of claim 14, said method further comprising incubating said one or more first nucleic acid molecules under conditions sufficient to make one or more second nucleic acid molecules complementary to all or a portion of said one or more first nucleic acid molecules.

17. A cDNA molecule made according to the method of claim 14.

18. A cDNA molecule made according to the method of claim 17.

19. A method for amplifying one or more nucleic acid molecules, said method comprising:
(a) mixing one or more nucleic acid templates with one or more inhibitors, one or more polypeptides having reverse transcription activity and one or more DNA polymerases; and
(b) incubating mixture of (a) under conditions sufficient to amplify one or more nucleic acid molecules complementary to all or a portion of said one or more templates.

20. A nucleic acid molecule amplified according to the method of claim 20.

21. A kit for use in reverse transcription, amplification, or sequencing of a nucleic acid molecule, said kit comprising one or more inhibitors.

22. The kit of claim 21, said kit further comprising one or more components selected from the group one or more nucleotides, one or more DNA polymerases, one or more buffers or buffering salts, one or more primers, one or more host cells, and one or more terminating agents.

23. A reaction mixture comprising:
(a) at least a 4000 micomolar concentration of one or more inhibitors; and
(b) one or more mRNA templates.

24. A composition comprising:
(a) at least a 4000 micomolar concentration of one or more inhibitors; and
(b) one or more mRNA templates.
